(19) United States
(12) Patent Application Publication
(10) Pub. No.: US 2026/0125536 A1
(43) Pub. Date: May 7, 2026

(54) NEW ANTIDEGRADANTS BASED ON FATTY ACIDS OR DERIVATIVES FUNCTIONALIZED WITH PHENYLENEDIAMINES

(71) Applicant: FLEXSYS AMERICA L.P., Akron, OH (US)

(72) Inventors: Jody Lee RODGERS, Gilmer, TX (US); Judicael Jacques CHAPELET, Akron, OH (US); Donald L. FIELDS, JR., Copley, OH (US); Frederick IGNATZ-HOOVER, Elyria, OH (US); Shanzuo JI, Beachwood, OH (US)

(21) Appl. No.: 18/686,212

(22) PCT Filed: Aug. 24, 2022
(86) PCT No.: PCT/US2022/075409

§ 371 Date: Feb. 23, 2024

Related U.S. Application Data
(60) Provisional application No. 63/236,280, filed on Aug. 24, 2021.

(51) Int. Cl. C08K 5/18; C07C 215/16; C07C 229/18

(52) U.S. Cl. CPC C08K 5/18; C07C 215/16; C07C 229/18; C07C 2601/16

(57) ABSTRACT

Antidegradant products are disclosed that may be made by a process comprising: hydroformylating one or more of: a fatty acid or fatty acid derivative having one or more unsaturations, e.g., carbon-to-carbon double bonds, and from 10 to 20 carbon atoms, or a fatty alcohol comprising one or more of a straight chain fatty alcohol having one or more unsaturations, e.g., unsaturated carbon-to-carbon double bonds, and from 10 to 20 carbon atoms, by reaction with carbon monoxide and hydrogen gases to obtain a hydroformylated intermediate; and functionalizing the hydroformylated intermediate by reaction with a substituted or unsubstituted paraphenylene diamine to obtain the antidegradant product.

### FIELD OF THE DISCLOSURE

The present disclosure provides fatty acids and derivatives functionalized with phenylenediamines, and methods of making them. These phenylenediamine compounds are particularly useful as antidegradants for rubber applications.

### BACKGROUND OF THE DISCLOSURE

Paraphenylene diamines are known to be useful as antidegradants. Thus, U.S. Pat. No. 3,409,586 discloses a diolefin rubber vulcanizate that contains an antiozonant amount of N-alkyl-N′-o-substituted-phenyl-para-phenylenediamine.

U.S. Pat. No. 5,117,063 discloses methods of producing 4-ADPA, wherein aniline and nitrobenzene are reacted under suitable conditions to produce 4-nitrodiphenylamine and/or 4-nitrosodiphenylamine and/or their salts, either or both of which are subsequently reduced to produce 4-ADPA. The 4-ADPA can be reductively alkylated to produce p-phenylenediamine products which are useful as antiozonants.

U.S. Pat. No. 6,140,538 discloses processes for preparing an optionally substituted 4-aminodiphenylamine comprising reacting an optionally substituted aniline and an optionally substituted nitrobenzene in the presence of water and a base while controlling the water content so as to ensure a molar ratio of water to the base charged of not less than about 4:1 at the start of the coupling reaction and not less than about 0.6:1 at the end of the coupling reaction to produce 4-nitrodiphenylamine and/or 4-nitrosodiphenylamine and/or salts thereof. The coupling reaction is followed by a hydrogenation reaction where the coupling reaction product is hydrogenated in the presence of a hydrogenation catalyst and added water so as to ensure a molar ratio of total water to base of at least about 4:1 at the end of hydrogenation. Aqueous and organic phases are obtained and the optionally substituted 4-aminodiphenylamine recovered from the organic phase.

WO 2017/112440A1 likewise discloses p-phenylenediamine compounds useful as antidegradants.

U.S. Pat. No. 5,134,200 describes polymers having chemically bound antidegradants that are prepared by reacting a polymer having olefinic unsaturation with carbon monoxide and hydrogen under hydroformylation conditions in the presence of a hydroformylation catalyst, an organic reaction solvent and a primary or secondary amine-containing antidegradant.

U.S. Pat. No. 7,576,227 describes processes of preparing industrial chemicals starting from seed oil feedstock compositions which includes metathesis to form a reduced chain unsaturated acid or ester which may be hydroformylated with reduction.

There remains a need in the art for antidegradants useful in rubber compositions, and especially those based on renewable raw materials.

### SUMMARY OF THE DISCLOSURE

In a first aspect, the present disclosure provides compounds represented by formula I:

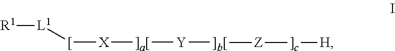

wherein
R¹ is a group selected from —OH, —O-alkyl, —O-aryl, —O-(alkylaryl), glycerol, glycerol monoester or diester, —NH₂, —NH(alkyl), —N(alkyl)₂, —NH(aryl), —N(aryl)₂, —NH(alkylaryl), —N(alkylaryl)₂, —SH, —S-alkyl, —S-aryl, or —S-(alkylaryl),
L¹ is a group selected from —(C═O)— or —(CH₂)—,
a+b+c is from 9 to 17,
and wherein X, Y, and Z are groups independently selected from methylene, alkenediyl, or substituted para-phenylenediamines corresponding to:

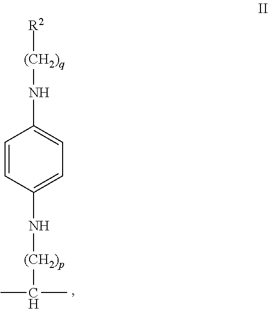

wherein
   p is 0 or 1,
   q is 0 or 1,
   the number of methylene groups is from 8 to 16,
   the number of substituted para-phenylenediamine groups is from 1 to 3,
   the number of alkenediyl groups is from 0 to 2; and
wherein R² is a group selected from —H, -alkyl, -aryl, -alkylaryl, -arylalkyl, or a moiety corresponding to:

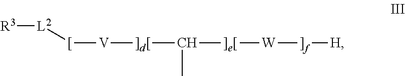

wherein
   R³ is a group selected from —OH, —O-alkyl, —O-aryl, —O-(alkylaryl), glycerol, glycerol monoester or diester, —NH₂, —NH(alkyl), —N(alkyl)₂, —NH(aryl), —N(aryl)₂, —NH(alkylaryl), —N(alkylaryl)₂, —SH, —S-alkyl, —S-aryl, or —S-(alkylaryl),
   L² is a group selected from —(C═O)— or —(CH₂)—,
   V and W are groups independently selected from methylene, alkenediyl, or substituted para-phenylenediamine,
   and wherein d+e+f is from 9 to 17.
      the number of methylene groups is from 8 to 16,
      the number of substituted para-phenylenediamine groups is from 0 to 3, and
      the number of alkenediyl groups is from 0 to 3.

In another aspect, the disclosure provides antidegradant products made by a process comprising hydroformylating one or more of a straight chain fatty acid having one or more unsaturations, e.g., carbon-to-carbon double bonds, and from 10 to 20 carbon atoms, or an amide or thioester or thiocarboxylic acid thereof, or a mono-, di-, or triglyceride thereof, or an alkyl ester or an aryl ester or an alkylaryl ester thereof wherein the alkyl or aryl or alkylaryl group has from one to twelve carbon atoms, or hydroformylating one or more of a straight chain fatty alcohol having one or more unsaturations, e.g., carbon-to-carbon double bonds, and from 10 to 20 carbon atoms by reaction with carbon monoxide and hydrogen gases to obtain a hydroformylated intermediate; and thereafter functionalizing the hydroformylated intermediate by reaction with a substituted or unsubstituted paraphenylene diamine to obtain the antidegradant product.

In a further aspect, the present disclosure provides compositions that comprise the compounds of formula I, for example vulcanizable elastomeric formulations, as well as articles made from them.

Further aspects of the disclosure are as set out below and in the claims that follow. It should be understood that the description and specific examples are intended for purposes of illustration only and are not intended to limit the spirit and scope of the present disclosure.

### DETAILED DESCRIPTION

Thus, in one embodiment, the disclosure provides compounds represented by formula I:

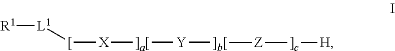

wherein
R¹ is a group selected from —OH, —O-alkyl, —O-aryl, —O-(alkylaryl), glycerol, glycerol monoester or diester, —NH₂, —NH(alkyl), —N(alkyl)₂, —NH(aryl), —N(aryl)₂, —NH(alkylaryl), —N(alkylaryl)₂, —SH, —S-alkyl, —S-aryl, or —S-(alkylaryl),
L¹ is a group selected from —(C═O)— or —(CH₂)—,
a+b+c is from 9 to 17,
and wherein X, Y, and Z are groups independently selected from methylene, alkenediyl, or substituted para-phenylenediamines corresponding to:

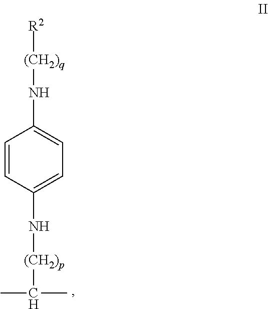

wherein
   p is 0 or 1,
   q is 0 or 1,
   the number of methylene groups is from 8 to 16,
   the number of substituted para-phenylenediamine groups is from 1 to 3,
   the number of alkenediyl groups is from 0 to 2; and
   wherein R² is a group selected from —H, -alkyl, -aryl, -alkylaryl, -arylalkyl, or a moiety corresponding to:

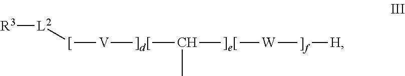

wherein
   R³ is a group selected from —OH, —O-alkyl, —O-aryl, —O-(alkylaryl), glycerol, glycerol monoester or diester, —NH₂, —NH(alkyl), —N(alkyl)₂, —NH(aryl), —N(aryl)₂, —NH(alkylaryl), —N(alkylaryl)₂, —SH, —S-alkyl, —S-aryl, or —S-(alkylaryl),
   L² is a group selected from —(C═O)— or —(CH₂)—,
   V and W are groups independently selected from methylene, alkenediyl, or substituted para-phenylenediamine,
   and wherein
   d+e+f is from 9 to 17.
   the number of methylene groups is from 8 to 16,
   the number of substituted para-phenylenediamine groups is from 0 to 3, and
   the number of alkenediyl groups is from 0 to 3.

In a second embodiment, according to the first embodiment, R¹ is a glycerol diester group, L¹ is —(C═O)—, the number of methylene groups is 16, the number of substituted para-phenylenediamine group is 1, the number of alkenediyl group is 0, p is 1, q is 0, and R² is a phenyl group.

In a third embodiment, R¹ is a glycerol diester group, L¹ is —(C═O)—, the number of methylene groups is 15, the number of substituted para-phenylenediamine groups is 2, the number of alkenediyl group is 0, p is 1, q is 0, and R² is a phenyl group.

In a fourth embodiment, R¹ is a glycerol diester group, L¹ is —(C═O)—, the number of methylene groups is 14, the number of substituted para-phenylenediamine groups is 1, the number of alkenediyl group is 1, p is 1, q is 0, and R² is a phenyl group.

In a fifth embodiment, R¹ is a glycerol diester group, L¹ is —(C═O)—, the number of methylene groups is 13, the number of substituted para-phenylenediamine groups is 2, the number of alkenediyl group is 1, p is 1, q is 0, and R² is a phenyl group.

In a sixth embodiment, R¹ is a —OH group, L¹ is —(C═O)—, the number of methylene groups is 16, the number of substituted para-phenylenediamine groups is 1, the number of alkenediyl group is 0, p is 1, q is 0, and R² is a methyl group.

In a seventh embodiment, R¹ and R³ are —OCH₃ groups, L¹ and L² are —(C═O)— groups, the number of methylene groups is 33, the number of substituted para-phenylenediamine group is 1, the number of alkenediyl group is 0, p is 1, and q is 1.

In an eighth embodiment, the disclosure provides antidegradant products made by a process comprising:
hydroformylating one or more of:
   a fatty acid or fatty acid derivative comprising one or more of a straight chain fatty acid having one or more unsaturations, e.g, carbon-to-carbon double bonds, and from 10 to 20 carbon atoms, or an amide or thioester or thiocarboxylic acid thereof, or a mono-, di-, or triglyceride thereof, or an alkyl ester or an aryl ester or an alkylaryl ester thereof wherein the alkyl or aryl or alkylaryl group has from one to twelve carbon atoms, or
   a fatty alcohol comprising one or more of a straight chain fatty alcohol having one or more unsaturations, e.g., carbon-to-carbon double bonds, and from 10 to 20 carbon atoms,
by reaction with carbon monoxide and hydrogen gases to obtain a hydroformylated intermediate; and
   functionalizing the hydroformylated intermediate by reaction with a substituted or unsubstituted paraphenylene diamine to obtain the antidegradant product.

In a ninth embodiment according to the eighth embodiment, the fatty acid or fatty acid derivative comprises soybean oil.

In a tenth embodiment, according to the eighth embodiment, the fatty acid or fatty acid derivative comprises one or more of soybean oil, olive oil, canola oil, corn oil, cottonseed oil, grapeseed oil, flax oil, hempseed oil, peanut oil, or sunflower oil.

In an eleventh embodiment, according to the eighth embodiment, the fatty acid or fatty acid derivative comprises a mono-, di-, or triglyceride of a residue of one or more of palmitic acid, stearic acid, oleic acid, linoleic acid, or linolenic acid.

In a twelfth embodiment, according to the eighth embodiment the fatty alcohol comprises one or more of palmitic alcohol, stearic alcohol, oleic alcohol, linoleic alcohol, or linolenic alcohol.

In a further embodiment, the disclosure provides a compound having the following formula:

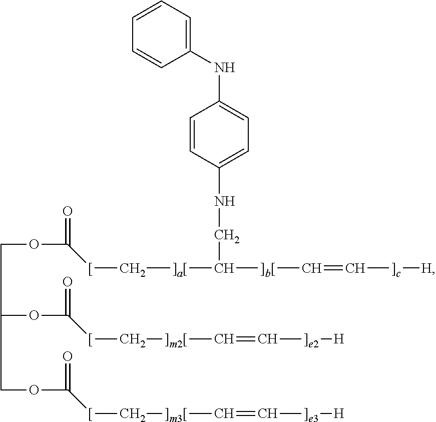

wherein m1 is from 11 to 17, e1 is from 0 to 3, m2 is from 11 to 17, e2 is from 0 to 3, m3 is from 11 to 17, e3 is from 0 to 3, and the groups —CH₂— and —CH═CH— can be in any order, and a, b, and c are described elsewhere, or a compound having formula X:

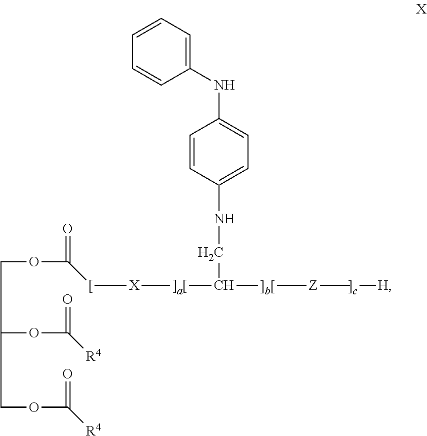

wherein each X and each Z are groups independently selected from methylene and alkenediyl, b is 1, and a+c is an integer from 9 to 17.

In a further embodiment, the disclosure provides a compound having the following formula:

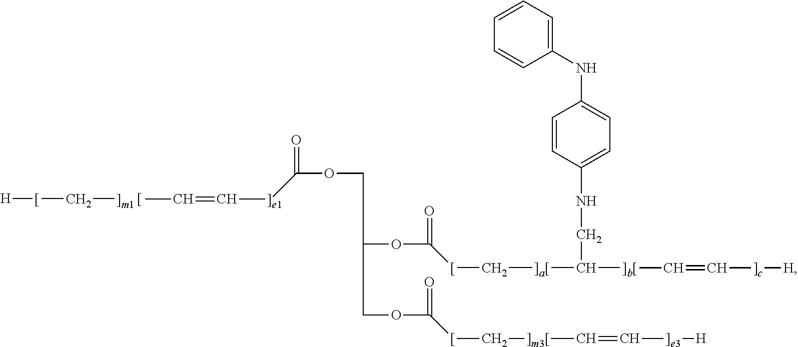

wherein m1 is from 11 to 17, e1 is from 0 to 3, m2 is from 11 to 17, e2 is from 0 to 3, m3 is from 11 to 17, e3 is from 0 to 3, and the groups —CH₂— and —CH═CH— can be in any order, and a, b, and c are described elsewhere, or a compound having formula XI:

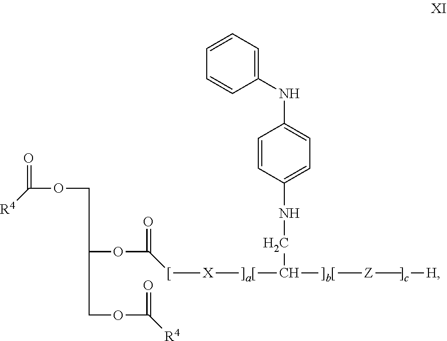

wherein each X and each Z are groups independently selected from methylene and alkenediyl, b is 1, and a+c is an integer from 9 to 17.

The disclosure also provides a compound having the following formula:

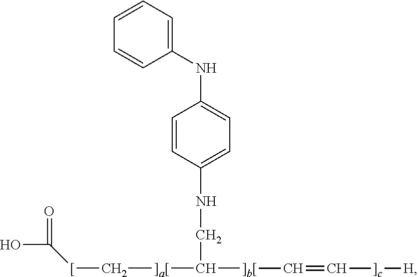

wherein a+b+c is from 9 to 17, or a compound having formula XII:

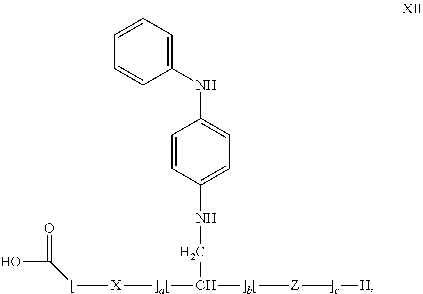

wherein each X and each Z are groups independently selected from methylene and alkenediyl, and b is 1, and a+c is an integer from 9 to 17.

In yet another aspect, the disclosure provides a compound having the following formula:

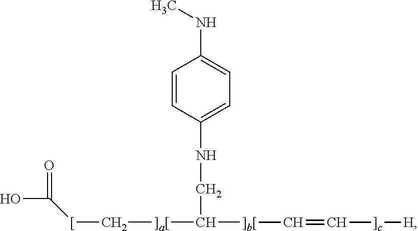

wherein a+b+c is from 9 to 17, or a compound having formula XIII:

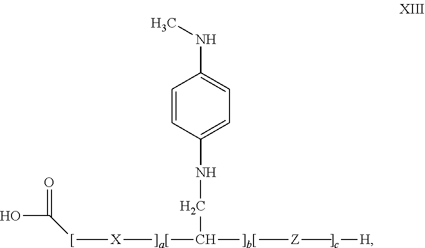

wherein each X and each Z are groups independently selected from methylene and alkenediyl, b is 1, and a+c is an integer from 9 to 17.

In yet another aspect, the disclosure provides a compound having the following formula:

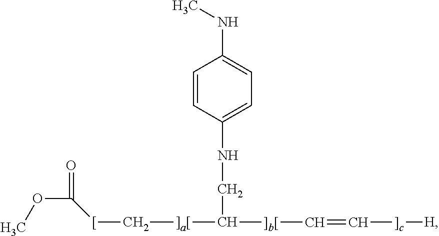

wherein a+b+c is from 9 to 17, or a compound having formula XIV:

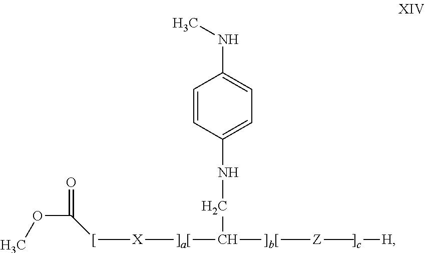

wherein each X and each Z are groups independently selected from methylene and alkenediyl b is 1, and a+c is an integer from 9 to 17.

In yet another aspect, the disclosure provides a compound having the following formula:

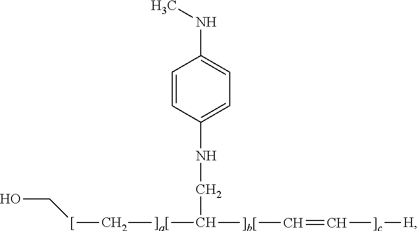

wherein a+b+c is from 9 to 17, or a compound having formula XV:

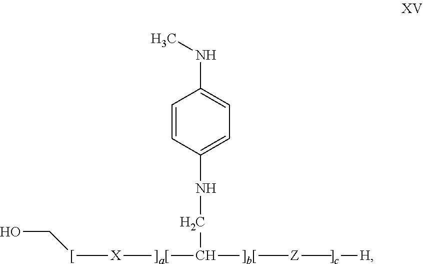

wherein each X and each Z are groups independently selected from methylene and alkenediyl b is 1, and a+c is an integer from 9 to 17.

In yet another aspect, the disclosure provides a compound having the following formula:

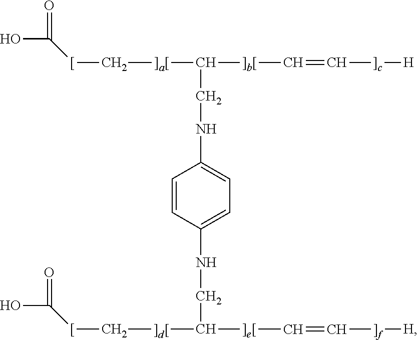

wherein a+b+c is from 9 to 17 and d+e+f is from 9 to 17, or a compound having formula XVI:

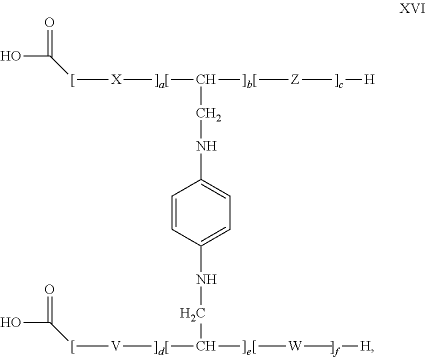

wherein each X and each Z are groups independently selected from methylene and alkenediyl b is 1, and a+c is an integer from 9 to 17, and
each V and each W are groups independently selected from methylene and alkenediyl e is 1, and d+f is an integer from 9 to 17.

In yet another aspect, the disclosure provides a compound having the following formula:

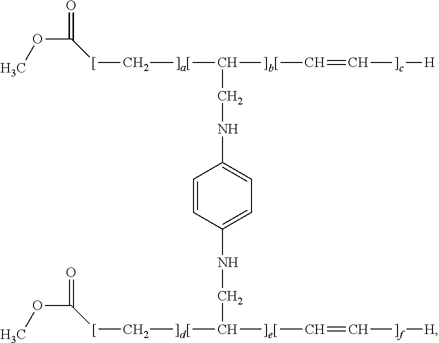

wherein a+b+c is from 9 to 17 and d+e+f is from 9 to 17, or a compound having formula XVII:

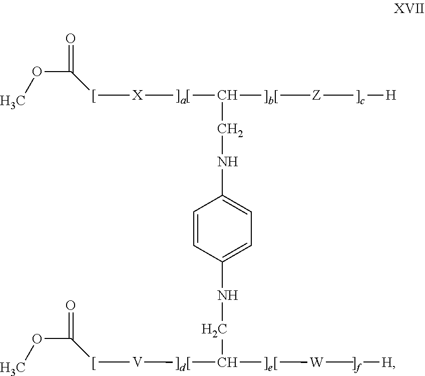

wherein each X and each Z are groups independently selected from methylene and alkenediyl b is 1, and a+c is an integer from 9 to 17, and
each V and each W are groups independently selected from methylene and alkenediyl e is 1, and d+f is an integer from 9 to 17.

In yet another aspect, the disclosure provides a compound having the following formula:

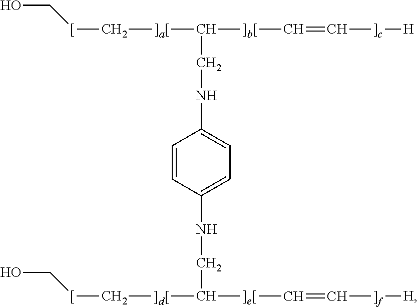

wherein a+b+c is from 9 to 17 and d+e+f is from 9 to 17, or a compound having formula XVIII:

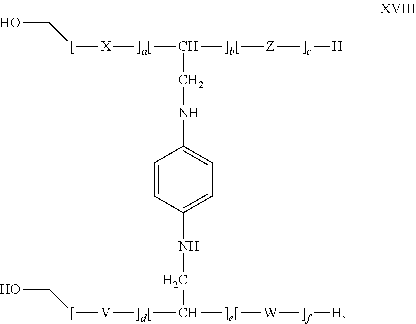

wherein each X and each Z are groups independently selected from methylene and alkenediyl b is 1, and a+c is an integer from 9 to 17, and
wherein each V and each W are groups independently selected from methylene and alkenediyl b is 1, and d+f is an integer from 9 to 17.

Thus, in one aspect, the disclosure provides the use of one or more fatty acids. The term “fatty acid” as used herein refers to a compound having a C₁₀-C₂₀ straight alkenyl chain terminated by a carboxylic acid or carboxylate functional group. The term “fatty acid derivative” as used herein refers to a fatty acid in which the carboxylic acid or carboxylate functional group has been transformed into an amide, a thioester, a thiocarboxylic acid, a thiocarboxylate, an alkyl ester, an aryl ester, an alkylaryl ester, or a compound resulting from the formation of an ester between the carboxylic acid or carboxylate group of the fatty acid and a hydroxy group of glycerol, a glycerol mono-ester, or a glycerol di-ester. The term “fatty alcohol” as used herein refers to a compound having a C₁₀-C₂₀ straight alkenyl chain terminated by a hydroxy group.

In another aspect, the disclosure provides compounds according to formula I, and their use as antidegradants. In this aspect, the compounds of formula I may be described herein as Compounds of the Disclosure. For example, Compounds of the Disclosure include, but are not limited to 4-aminodiphenylamine (4-ADPA) functionalized with soybean oil, 4-ADPA functionalized with fatty acids, 4-(methylamino)phenylamine (4-MAPA) functionalized with fatty acids, 4-MAPA functionalized with fatty alcohols.

As utilized herein, the following terms or phrases are defined as follows:

“Antidegradant” refers to a material that inhibits degradation (as caused by for example, through heat, light, oxidation, and/or ozonation), or manifestations thereof, of a composition, formulation or article to which it is added or applied.

“Antifatigue agent” refers to a material that improves the flex fatigue resistance of a composition, formulation or article to which it is added or applied after a period of in-service application time whereby the composition, formulation or article is subjected to thermal, oxidative, ozone and mechanical degradative forces.

“Antioxidant” refers to a material that inhibits oxidative degradation of a composition, formulation or article to which it is added or applied.

“Antiozonant” refers to a material that inhibits ozone exposure degradation of a composition, formulation or article to which it is added or applied.

“Elastomer” means any polymer which after vulcanization (or crosslinking) and at room temperature can be stretched under low stress, for example to at least twice its original length and, upon immediate release of the stress, will return with force to approximately its original length, including without limitation rubber.

“Vulcanizable Elastomeric Formulation” means a composition that includes an elastomer and that is capable of vulcanization when placed under vulcanization conditions.

The term “alkyl” as used herein by itself or as part of another group refers to a straight- or branched-chain aliphatic hydrocarbon containing one to twenty-six carbon atoms, i.e., a C₁-C₂₆ alkyl, or the number of carbon atoms designated, e.g., C₁-C₃ alkyl such as methyl, ethyl, propyl, or isopropyl; a C₁-C₄ alkyl such as methyl, ethyl, propyl, isopropyl, n-butyl, isobutyl, sec-butyl, or t-butyl; and so on. In one embodiment the alkyl is a straight-chain alkyl. In another embodiment, the alkyl is a branched-chain alkyl. In one embodiment, the alkyl is a C₁-C₈ alkyl. In another embodiment, the alkyl is a C₉-C₁₇ alkyl. In another embodiment, the alkyl is a C₁₀-C₂₂ alkyl.

The term “alkenyl” as used herein by itself or as part of another group refers to a C₂-C₂₆ alkyl group containing at least one carbon-to-carbon double bond, e.g., one, two, three, or four carbon-to-carbon double bonds. In one embodiment, the alkenyl group is a C₅-C₂₆ alkenyl group. In another embodiment, the alkenyl group is a C₇-C₁₉ alkenyl group. In another embodiment, the alkenyl group has one carbon-to-carbon double bond. In another embodiment, the alkenyl group has two carbon-to-carbon double bonds. In another embodiment, the alkenyl group has three carbon-to-carbon double bonds. In another embodiment, the alkenyl group has four carbon-to-carbon double bonds.

The term “aryl” as used herein by itself or as part of another group refers to an aromatic ring system having six to fourteen carbon atoms, i.e., C₆-C₁₄ aryl. Non-limiting exemplary aryl groups include phenyl (abbreviated as “Ph”), naphthyl, phenanthryl, anthracyl, indenyl, azulenyl, biphenyl, biphenylenyl, and fluorenyl groups. In one embodiment, the aryl group is phenyl or naphthyl. In another embodiment, the aryl group is phenyl.

The term “alkylaryl” as used herein by itself or as part of another group refers to an alkyl group that is substituted with one or more aryl groups. In another embodiment, the alkylaryl group is a benzyl group, which has the following structure:

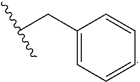 [SMILES: *Cc1ccccc1]

The term “arylalkyl” as used herein by itself or as part of another group refers to an aryl group that is substituted with one or more alkyl groups. In another embodiment, the arylalkyl group is a p-tolyl group, which has the following structure:

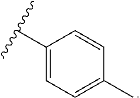 [SMILES: *c1ccc(C)cc1 ; "."]

The term “methylene” as used herein by itself or as part of another group refers to a —CH₂— group.

The term “alkenediyl” as used herein by itself or as part of another group refers to a —CH═CH— group.

The term “glycerol” refers to the following compound:

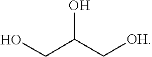

The term “glycerol mono-ester” refers to compounds having one of the following formulae:

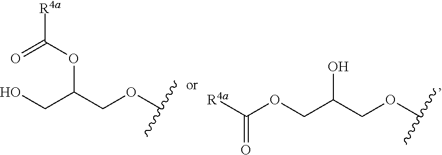

wherein each R^{4a} is independently selected from the group consisting of C₅₋₂₆ alkyl and C₅₋₂₆ alkenyl.

The term “glycerol di-ester” refers to compounds having one of the following formulae:

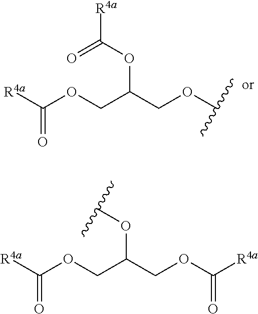

wherein each R^{4a} is independently selected from the group consisting of C₅₋₂₆ alkyl and C₅₋₂₆ alkenyl.

The term “hydroxy” as herein used by itself or as part of another group refers to —OH.

The term “amide” as herein used by itself or as part of another group refers to —C(═O)NH—.

The term “ester” as herein used by itself or as part of another group refers to —C(═O)O—, wherein the terminal oxygen atom is not bonded to a hydrogen atom. The term “alkyl ester” refers to an ester wherein the terminal oxygen atom is attached to an alkyl group. The term “aryl ester” refers to an ester wherein the terminal oxygen atom is attached to an aryl group. The term “alkylaryl ester” refers to an ester wherein the terminal oxygen atom is attached to an alkylaryl group.

The term “thioester” as herein used by itself or as part of another group refers to —C(═O)S—, wherein the sulfur atom is not bonded to a hydrogen atom. The term “alkyl thioester” refers to a thioester wherein the terminal sulfur atom is attached to an alkyl group. The term “aryl thioester” refers to a thioester wherein the terminal sulfur atom is attached to an aryl group. The term “alkylaryl thioester” refers to a thioester wherein the terminal sulfur atom is attached to an alkylaryl group.

The term “thiocarboxylic acid” as herein used by itself or as part of another group refers to —C(═O)SH.

The term “thiocarboxylate” as herein used by itself or as part of another group refers to —C(═O)S—.

The term “carboxylic acid” as herein used by itself or as part of another group refers to —C(═O)OH.

The term “carboxylate” as herein used by itself or as part of another group refers to —C(═O)O—.

Compounds of the Disclosure are advantageously believed to demonstrate antioxidant and antiozonant properties for rubber articles.

In one aspect, the present disclosure provides compounds according to the following formula I:

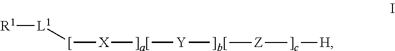

wherein
R¹ is a group selected from —OH, —O-alkyl, —O-aryl, —O-(alkylaryl), glycerol, glycerol monoester or diester, —NH₂, —NH(alkyl), —N(alkyl)₂, —NH(aryl), —N(aryl)₂, —NH(alkylaryl), —N(alkylaryl)₂, —SH, —S-alkyl, —S-aryl, or —S-(alkylaryl),
L¹ is a group selected from —(C═O)— or —(CH₂)—,
a+b+c is from 9 to 17,
and wherein X, Y, and Z are groups independently selected from methylene, alkenediyl, or substituted para-phenylenediamines corresponding to:

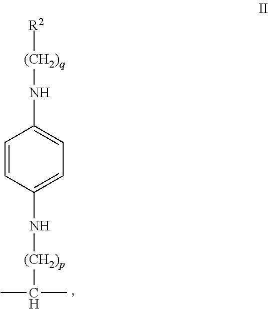

wherein
   p is 0 or 1,
   q is 0 or 1,
   the number of methylene groups is from 8 to 16,
   the number of substituted para-phenylenediamine groups is from 1 to 3,
   the number of alkenediyl groups is from 0 to 2; and
wherein R² is a group selected from —H, -alkyl, -aryl, -alkylaryl, -arylalkyl, or a moiety corresponding to:

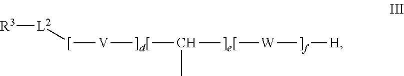

wherein
   R³ is a group selected from —OH, —O-alkyl, —O-aryl, —O-(alkylaryl), glycerol, glycerol monoester or diester, —NH₂, —NH(alkyl), —N(alkyl)₂, —NH(aryl), —N(aryl)₂, —NH(alkylaryl), —N(alkylaryl)₂, —SH, —S— alkyl, —S-aryl, or —S-(alkylaryl),
   L² is a group selected from —(C═O)— or —(CH₂)—,
   V and W are groups independently selected from methylene, alkenediyl, or substituted para-phenylenediamine,
   and wherein d+e+f is from 9 to 17.
      the number of methylene groups is from 8 to 16,
      the number of substituted para-phenylenediamine groups is from 0 to 3, and
      the number of alkenediyl groups is from 0 to 3.

Thus, Compounds of the Disclosure include those set out in formula I above.

In one aspect, R¹ may be a group selected from —OH, —O-alkyl, —O-aryl, —O-(alkylaryl), glycerol, glycerol monoester or diester, —NH₂, —NH(alkyl), —N(alkyl)₂, —NH(aryl), —N(aryl)₂, —NH(alkylaryl), —N(alkylaryl)₂, —SH, —S-alkyl, —S-aryl, or —S-(alkylaryl). For example, R¹ may be a glycerol ester in which three fatty acid derivatives are ultimately present on the molecule, when the L¹ functionality is —(C═O)—. Alternatively, R¹ may be hydroxyl, in which case Compounds of the Disclosure are simply fatty acid derivatives (when L¹ is —(C═O)—), (when L¹ is —(CH₂)—). Alternatively, R¹ may be a methyl ester in which case Compounds of the Disclosure are simply fatty monoester derivatives (when L¹ is —(C═O)—).

In some aspects, the groups X, Y, and Z are independently selected from methylene, alkenediyl, or substituted para-phenylenediamines corresponding to:

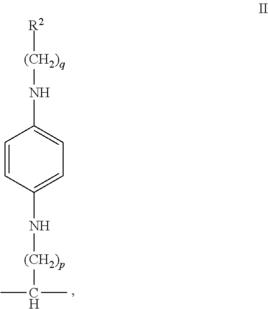

In this aspect, these groups can be in any order, so long as a+b+c is, for example, from 9 to 17. This mixture of groups results, of course, from the use of fatty acids or fatty alcohols and derivatives thereof as the raw materials. There may thus be remaining unsaturations, e.g., carbon-to-carbon double bonds, as well as multiple substituted para-phenylenediamine groups along the chain of the fatty acids or fatty alcohols. According to formula II, then, p can be 0 or 1, and q can be 0 or 1.

Because Compounds of the Disclosure are derived from fatty acids or fatty alcohols and derivatives thereof, the number of methylene groups will be, for example, from 8 to 16, the number of substituted para-phenylenediamine groups will be, for example, from 1 to 3, and the number of alkenediyl groups is from 0 to 2.

Further according to formula II, R² may be a group selected, for example, from —H, -alkyl, -aryl, -alkylaryl, or -arylalkyl, in which case Compounds of the Disclosure may comprise a single fatty acid, a fatty acid derivative thereof, or a fatty alcohol.

Alternatively, R² may be a moiety corresponding to:

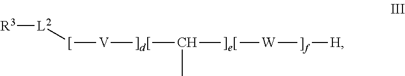

in which
R³ is a group selected from —OH, —O-alkyl, —O-aryl, —O-(alkylaryl), glycerol, glycerol monoester or diester, —NH₂, —NH(alkyl), —N(alkyl)₂, —NH(aryl), —N(aryl)₂, —NH(alkylaryl), —N(alkylaryl)₂, —SH, —S-alkyl, —S-aryl, or —S-(alkylaryl),
L² is a group selected from —(C═O)— or —(CH₂)—,
V and W are groups independently selected from methylene, alkenediyl, or substituted para-phenylenediamine,
   and wherein d+e+f is from 9 to 17.
the number of methylene groups is from 8 to 16,
the number of substituted para-phenylenediamine groups is from 0 to 3, and
the number of alkenediyl groups is from 0 to 3.

In this aspect, one fatty acid, derivative thereof or fatty alcohol is essentially linked with another one. It will thus be clear that formula I is, in that sense, analogous to figure III, and is linked by means of the paraphenylene diamine derivative of formula II.

In some aspects, fatty acids, fatty acid derivatives, and fatty alcohols and derivatives are used as raw materials, as already described. Derivatives thus include triglycerides, diglycerides, monoglycerides, fatty acids, fatty carboxylate salts, fatty monoesters, and fatty alcohols having between 15 and 18 carbon atoms per chain, such as the palmitic, stearic, oleic, linoleic, linolenic derivatives, and the like.

In some aspects, Compounds of the Disclosure may be produced in different ways. For instance, an unsaturated triglyceride such as soybean oil may be reacted with a mixture of carbon monoxide and hydrogen gases under pressure to form an aldehyde-containing intermediate, for example hydroformylated soybean oil. The aldehyde moiety in hydroformylated soybean oil may condense with the primary amine in a para-substituted aniline, for example 4-aminodiphenylamine (4-ADPA) to form an imine-containing intermediate, for example 4-ADPA and hydroformylated soybean oil condensation product. The imine moiety in the condensation product may be reacted with a reducing agent, for example hydrogen gas or sodium triacetoxyborohydride (STAB) to obtain the desired compounds of formula I as 4-ADPA functionalized with soybean oil.

In another instance, an unsaturated triglyceride such as soybean oil may be reacted with a mixture of carbon monoxide and hydrogen gases under pressure to form an aldehyde-containing intermediate, for example hydroformylated soybean oil. The aldehyde moiety in hydroformylated soybean oil may condense with the primary amine in a para-substituted aniline, for example 4-ADPA to form an imine-containing intermediate, for example 4-ADPA and hydroformylated soybean oil condensation product. The imine moiety in the condensation product may be reacted with a reducing agent, for example hydrogen gas or STAB to form 4-ADPA functionalized with soybean oil having intact triglyceridic esters. The esters moieties in 4-ADPA functionalized with soybean oil can be hydrolyzed or saponified, for example with sodium hydroxide to obtain the desired compounds of formula I as the 4-ADPA functionalized with fatty sodium carboxylate salts, or, upon protonation as the 4-ADPA functionalized with fatty carboxylic acids.

In another instance, an unsaturated triglyceride such as soybean oil may be hydrolyzed to form a mixture of fatty acid intermediates. The alkene moiety in the fatty acid intermediates may be reacted with a mixture of carbon monoxide and hydrogen gases under pressure to form an aldehyde-containing intermediate, for example hydroformylated fatty acids. The aldehyde moiety in hydroformylated fatty acids may condense with the primary amine in a para-substituted aniline, for example 4-(methylamino)phenylamine (4-MAPA) to form an imine-containing intermediate, for example 4-MAPA and hydroformylated fatty acid condensation product. The imine moiety in the condensation product may be reacted with a reducing agent, for example hydrogen gas or STAB to obtain the desired compounds of formula I as the 4-MAPA functionalized with fatty acids.

In another instance, soybean oil might be trans-esterified with methanol, ethanol, propanol, or any other suitable short alcohols to form a mixture of monoester of fatty acid intermediates. The alkene moiety in the monoester of fatty acid intermediates might be hydroformylated to form an aldehyde-containing intermediate. The aldehyde-containing intermediate might be reductively alkylated with a phenylenediamine possessing one primary amine, for example 4-ADPA to obtain the desired compound of formula I as, for example, the 4-ADPA functionalized with monoester of fatty acids.

In another instance, soybean oil might be reduced to form a mixture of fatty alcohol intermediates. The alkene moiety in the fatty alcohol intermediates might be hydroformylated to form an aldehyde-containing intermediate. The aldehyde-containing intermediate might be reductively alkylated with a phenylenediamine possessing one primary amine, for example 4-ADPA to obtain the desired compound of formula I as, for example, the 4-ADPA functionalized with fatty alcohols.

In another instance, para-nitroaniline (PNA) might be reduced and reductively alkylated with the intermediate hydroformylated soybean oil, hydroformylated fatty acid, hydroformylated monoester of fatty acid, or hydroformylated fatty alcohol to obtain the desired compound of formula I as the phenylenediamine linked to two soybean oil moieties, two fatty acid moieties, two monoester of fatty acid moieties, or two fatty alcohol moieties respectively.

The unsaturated triglyceride may correspond, for example, to the compounds of formula IV:

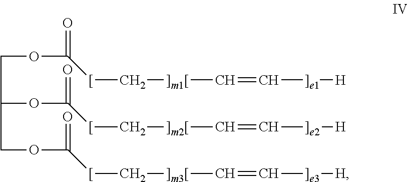

wherein m1 is from 11 to 17, e1 is from 0 to 3, m2 is from 11 to 17, e2 is from 0 to 3, m3 is from 11 to 17, e3 is from 0 to 3, and the groups —CH₂— and —CH═CH— can be in any order. We have found soybean oil to be a suitable unsaturated triglyceride. However, other suitable unsaturated triglycerides may be used, such as olive oil, canola oil, corn oil, cottonseed oil, grapeseed oil, flax oil, hempseed oil, peanut oil, sunflower oil, and the like.

In another embodiment, partial or complete hydrolysis of the three esters contained in a triglyceride oil may be carried out in boiling alcoholic solvent with the use of a salt containing the hydroxide anion to obtain fatty acid derivatives disclosed herein. We have found isopropanol (IPA) and sodium hydroxide to be suitable solvent and hydroxide salt respectively to carry out the hydrolysis. Other suitable solvents include methanol, ethanol, n-propanol, n-butanol, sec-butanol, tert-butanol, and the like. Other suitable hydroxide salts include those in which the cation is potassium, lithium, rubidium, or cesium, as well as ammonium or alkyl ammoniums derived by addition of proton(s) to a nitrogenous base.

The unsaturated diglycerides thus obtained may correspond, for example, to the compounds of formula IVa and IVb:

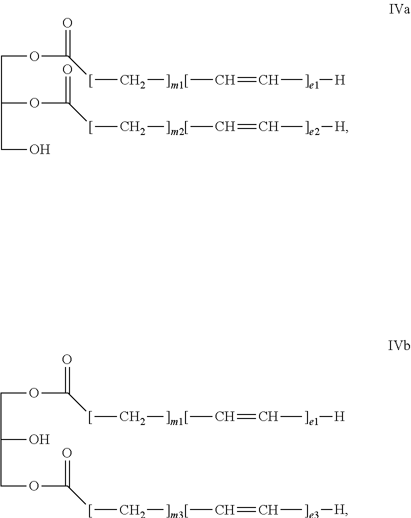

wherein m1, e1, m2, e2, m3, and e3 are as already described hereinbefore and with respect to formula IV.

Under harsher conditions (additional amount of hydroxide salt), the unsaturated monoglyceride obtained may correspond, for example, to the compounds of formula IVc and IVd:

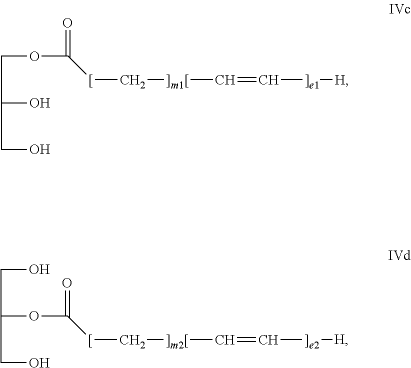

wherein m1, e1, m2, and e2 are likewise as already described.

Under the harshest conditions (excess amount of hydroxide salt), the mixture of fatty carboxylic acids obtained upon acidic workup may correspond, for example, to the compounds of formula IVe:

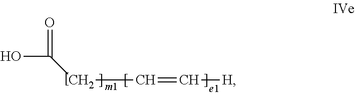

wherein m1 and e1 are likewise as already described. Glycerol by-produced from this reaction can be removed or kept as physically blended with the compounds of formula IVe for further chemical transformations. The fatty acids may thus include significant amounts of glycerol or products of further glycerol reactions. Fatty acids include stearic acid, oleic acid, linolenic acid, and linoleic acid.

In another embodiment, trans-esterification of the unsaturated triglyceride corresponding to the compounds of formula IV may be carried out using appropriate amount and type of alcohol in presence of appropriate amount and type of catalyst. The unsaturated fatty acid monoesters thus obtained may correspond, for example, to the compounds of formula IVf:

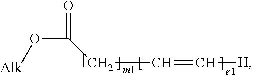

wherein m1 and e1 are likewise as already described, and Alk includes short hydrocarbon moieties such as methyl, ethyl, isopropyl, propyl, butyl, sec-butyl, tert-butyl, and the like. Glycerol by-produced from this reaction can be removed or kept as physically blended with the compounds of formula IVf for further chemical transformations as described above.

In another embodiment, reduction of the three esters in unsaturated triglyceride corresponding to the compounds of formula IV may be carried out with appropriate amount and type of reducing agents. The fatty alcohols thus obtained may correspond, for example, to the compounds of formula IVg:

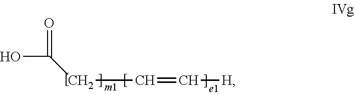

wherein m1 and e1 are likewise as already described. Glycerol by-produced from this reaction can be removed or kept as physically blended with the compounds of formula IVg for further chemical transformations.

In some aspects, hydroformylation of alkenes contained in any of the compounds of formulas IV to IVg may be carried out with a mixture of carbon monoxide and hydrogen gases under pressure. We have found a 1:1 molar ratio of carbon monoxide to hydrogen to be a suitable ratio to conduct the hydroformylation. However, variable molar ratios of carbon monoxide and hydrogen may be used, ranging from 0.5 to 5.0.

The “hydroformylation catalyst metal” may be selected from the Group VIII transition metals and may be provided in the form of various metal compounds such as carboxylate salts of the transition metal. Rhodium is the preferred Group VIII metal. The source of rhodium for the active catalyst includes rhodium(II) or rhodium(III) salts of carboxylic acids, examples of which include di-rhodium tetraacetate dihydrate, rhodium(II) acetate, rhodium(II) isobutyrate, rhodium(II) 2-ethylhexanoate, rhodium(II) benzoate and rhodium(II) octanoate. Also, rhodium carbonyl species such as Rh₄(CO)₁₂, Rh₆(CO)₁₆ and rhodium(I) acetylacetonate dicarbonyl may be suitable sources of rhodium. Additionally, rhodium organophosphine complexes such as tris(triphenylphosphine) rhodium carbonyl hydride may be used when the phosphine moieties of the complex feed are easily displaced. Other rhodium sources include rhodium salts of strong mineral acids such as chlorides, bromides, nitrates, sulfates, phosphates and the like. Rhodium bis-2-ethylhexanoate is a particularly preferred source of rhodium from which to prepare the complex catalyst because it is a convenient source of soluble rhodium, as it can be efficiently prepared from inorganic rhodium salts such as rhodium halides.

The “hydroformylation catalyst ligand” is defined as an organophosphorus molecule that is capable of coordinating to the hydroformylation catalyst metal. Catalyst ligands and their methods of preparation are well known in the art with common examples being organophosphines (monomeric as well as chelating forms), organophosphites (monomeric as well as chelating forms), halophosphites and phosphoramidites. We have found triphenylphosphine (TPP), tricyclohexylphosphine (TCHP), tris(2,4-di-tert-butylphenyl) phosphite (TDBP), and 2,2′-bis((diphenylphosphaneyl)methyl)-1,1′-biphenyl (BISBI)

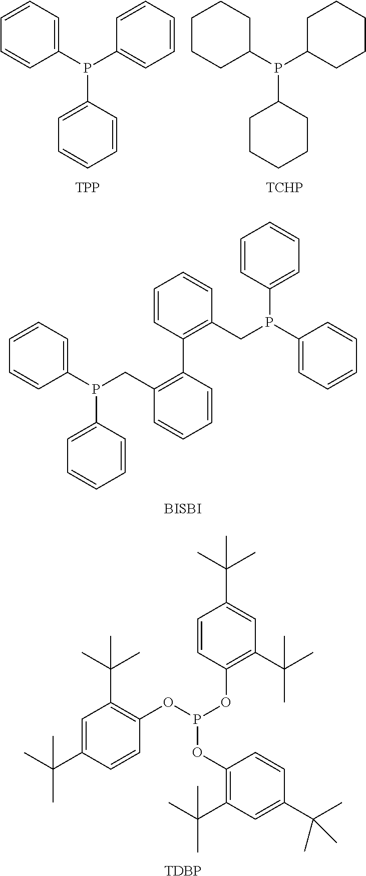

to be suitable ligands to conduct the hydroformylation. Other suitable ligands include, without limitation, trimethylphosphine, triethylphosphine, 1,2-bis(diphenylphosphino)ethane, 1,2-bis(dimethylphosphino)ethane, bis(diphenylphosphinoethyl)phenylphosphine, and the like.

The “hydroformylation catalyst compound” is defined as the combination of hydroformylation catalyst metal and hydroformylation catalyst ligand to form the active hydroformylation catalyst. In general, hydroformylation reactions involve the production of aldehydes by reacting an olefinic unsaturated compound with carbon monoxide and hydrogen in the presence of a catalyst compound homogeneously dissolved in a liquid medium that also contains a solvent for the catalyst, and free catalyst ligand (i.e., ligand that is not complexed with the rhodium metal in the active complex catalyst) that is likewise homogeneously dissolved. The hydroformylation process may be carried out in any excess amount of free catalyst ligand desired with at least one mole of free catalyst ligand per mole catalyst metal present in the reaction medium. In general, amounts of catalyst ligand of from about 4 to about 50 moles per mole rhodium present in the reaction medium should be suitable for most purposes, said amounts being the sum of both the amount of catalyst ligand that is bound (complexed) to the rhodium present and the amount of free (non-complexed) catalyst ligand present. Of course, if desired, make-up catalyst ligand can be supplied to the reaction medium of the hydroformylation process, at any time and in any suitable manner, to maintain a predetermined level of free catalyst ligand in the reaction medium. Moreover, it is to be understood that while the catalyst ligand of the catalyst compound and excess free catalyst ligand in a given process are both normally the same, different catalyst ligands, as well as mixtures of two or more different catalyst ligands, may be employed for each purpose in any given process, if desired.

Examples of hydroformylation solvents include, but are not limited to, alkanes, cycloalkanes, alkenes, cycloalkenes, carbocyclic aromatic compounds, alcohols, esters, ketones, acetals, ethers and water. Specific examples of such solvents include alkane and cycloalkanes such as dodecane, decalin, octane, iso-octane mixtures, cyclohexane, cyclooctane, cyclododecane, methylcyclohexane; aromatic hydrocarbons such as benzene, toluene, xylene isomers, tetralin, cumene, alkyl-substituted aromatic compounds such as the isomers of diisopropylbenzene, triisopropylbenzene and tert-butylbenzene; crude hydrocarbon mixtures such as naphtha, mineral oils and kerosene; high-boiling esters such as 2,2,4-trimethyl-1,3-pentanediol diisobutyrate or dioctylterephthalate. The starting olefin and/or aldehyde product of the hydroformylation process also may be used. The main criteria for the solvent is that it dissolves the catalyst and olefin substrate and does not act as a poison to the catalyst.

In some aspects, one or more of a straight chain fatty acid having one or more unsaturations, e.g., carbon-to-carbon double bonds, and from 10 to 20 carbon atoms, or an amide or thioester or thiocarboxylic acid thereof, or a mono-, di-, or triglyceride thereof, or an alkyl ester or an aryl ester or an alkylaryl ester thereof wherein the alkyl or aryl or alkylaryl group has from one to twelve carbon atoms; or one or more of a straight chain fatty alcohol having one or more unsaturations, e.g., carbon-to-carbon double bonds, and from 10 to 20 carbon atoms, may be hydroformylated by reaction with carbon monoxide and hydrogen gas, to obtain a hydroformylated intermediate.

The aldehyde-containing intermediate thus obtained may correspond, for example, to formula V:

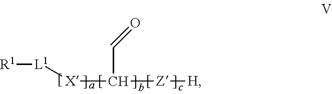

wherein R¹, L¹, a, b, and c are as already described hereinbefore and with respect to formula I, and wherein X′ and Z′ are groups independently selected from methylene or alkenediyl. Therefore, such aldehyde-containing intermediates can be synthesized by hydroformylation of the fatty acids and fatty acid derivatives described herein, whether unsaturated triglycerides, unsaturated diglycerides, unsaturated monoglycerides, unsaturated fatty acids, unsaturated fatty carboxylate salts, unsaturated monoester of fatty acids; or unsaturated fatty alcohols.

The para-substituted primary aniline, may correspond, for example, to the compounds of formula VI:

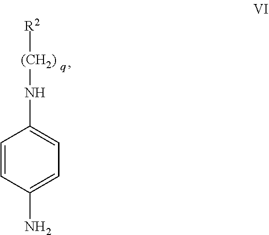

wherein R² and q are as already described hereinbefore and with respect to formula II. We have found 4-aminodiphenylamine (4-ADPA), and 4-(methylamino)phenylamine (4-MAPA)

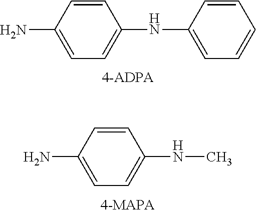

to be suitable para-substituted primary anilines. Other suitable para-substituted primary anilines include, without limitation, N-ethyl-4-aminoaniline, N-propyl-4-aminoaniline, N-butyl-4-aminoaniline, N-pentyl-4-aminoaniline, N-hexyl-4-aminoaniline, 4-hydroxyaniline, N-tolyl-para-phenylenediamine, N-naphtalenyl-para-phenylenediamine, N-biphenyl-para-phenylenediamine, and the like. These compounds are also referred to herein as “substituted or unsubstituted para-phenylene diamines.” Additionally, para-nitroaniline (PNA)

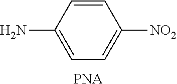

is a suitable para-substituted anilines as the nitro group can undergo reduction to form the phenylenediamine subunit.

The condensation of the aldehyde-containing intermediate with the para-substituted primary aniline thus furnishes an imine-containing intermediate, which may correspond, for example, to formula VII:

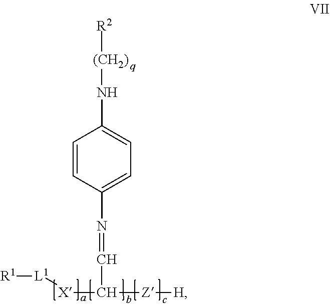

wherein R¹, L¹, X′, Z′, a, b, c, q, and R² are likewise as already described hereinbefore and with respect to formula V and formula II.

Alternatively, the condensation of the aldehyde-containing intermediate with PNA furnishes an imine-containing intermediate, which may correspond, for example, to formula VIII or formula IX:

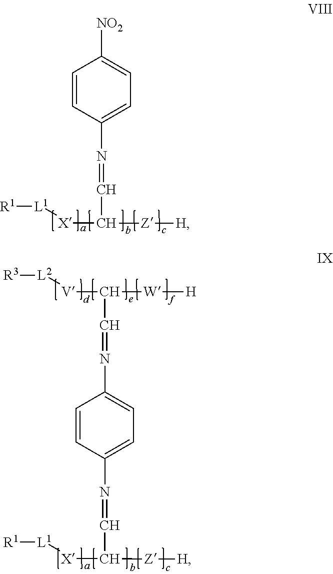

wherein R¹, L¹, X′, Z′, a, b, and c are likewise as already described hereinbefore and with respect to formula VII, wherein R³, L², d, e, and f are likewise as already described hereinbefore and with respect to formula III, and wherein V and W are groups independently selected from methylene or alkenediyl.

The imine-containing intermediate may be reduced in different ways to afford the desired compounds of formula I. For instance, we have found hydrogen gas under pressure in presence of catalytic amounts of platinum on carbon in hot toluene to be a suitable reactive system to reduce the imine-containing intermediate to the amines of formula I. Other catalysts based on transition metals might be used to enable the hydrogenation, such as palladium, iridium, ruthenium, and the like. In another instance, we have found sodium triacetoxyborohydride (STAB) in isopropanol solvent at room temperature to be a suitable reactive system to reduce some imine-containing intermediates to amines of formula I. Other suitable reducing agents based on hydride compounds might be used, such as sodium cyanoborohydride and the like.

In another embodiment, we were able to convert a phenylenediamine functionalized with triglyceride to phenylenediamine functionalized with fatty carboxylate salts (and their fatty acid counterparts upon acidic workup) by hydrolysis of the three triglyceridic esters. We have found sodium hydroxide in boiling isopropanol solvent to be a suitable reactive system to hydrolyze the 4-ADPA functionalized with triglyceridic ester to 4-ADPA functionalized with fatty carboxylate salts. Upon protonation with aqueous strong acid such as hydrochloric acid, we were able to produce compounds of formula I as 4-ADPA functionalized with fatty carboxylic acids physically blended with unfunctionalized fatty carboxylic acids. In other words, the fatty acids containing no 4-ADPA moieties were not separated from the 4-ADPA functionalized with fatty acids.

In another embodiment, a phenylenediamine functionalized with triglyceride may be converted to a phenylenediamine functionalized with fatty amides by aminolysis with ammonia or an amine.

In another embodiment, we were able to synthesize a phenylenediamine functionalized with fatty acid via an alternative route: soybean oil was hydrolyzed with excess sodium hydroxide in boiling isopropanol (IPA) to form a mixture of unsaturated and saturated fatty carboxylate salts. Upon acidic workup, a mixture of fatty acids was obtained as intermediate. Then, the alkene moieties in the unsaturated fatty acid intermediates were reacted with a mixture of carbon monoxide and hydrogen gases under pressure to form a mixture of fatty acids, some of them being hydroformylated. Then, the aldehyde moiety in hydroformylated fatty acids was condensed with 4-MAPA to form an imine-containing intermediate. Finally, the imine moiety in the condensation product was reacted with a reducing agent, for example hydrogen gas under pressure or STAB to obtain the desired compounds of formula I as the 4-MAPA functionalized with fatty acids physically blended with unfunctionalized fatty carboxylic acids. In other words, the fatty acids containing no 4-MAPA moieties were not separated from the 4-MAPA functionalized with fatty acids.

The disclosure also provides the following embodiments.

Embodiment I. A compound represented by formula I:

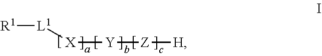

wherein
R¹ is a group selected from —OH, —O-alkyl, —O-aryl, —O-(alkylaryl), glycerol, glycerol monoester or diester, —NH₂, —NH(alkyl), —N(alkyl)₂, —NH(aryl), —N(aryl)₂, —NH(alkylaryl), —N(alkylaryl)₂, —SH, —S-alkyl, —S-aryl, or —S-(alkylaryl),
L¹ is a group selected from —(C═O)— or —(CH₂)—,
a+b+c is from 9 to 17,
and wherein X, Y, and Z are groups independently selected from methylene, alkenediyl, or substituted para-phenylenediamines corresponding to:

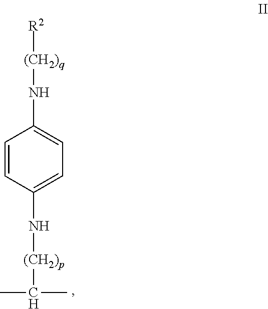

wherein
p is 0 or 1,
q is 0 or 1,
the number of methylene groups is from 8 to 16,
the number of substituted para-phenylenediamine groups is from 1 to 3,
the number of alkenediyl groups is from 0 to 2; and
wherein R² is a group selected from —H, -alkyl, -aryl, -alkylaryl, -arylalkyl, or a moiety corresponding to:

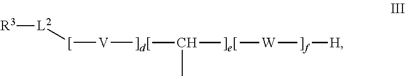

wherein
R³ is a group selected from —OH, —O-alkyl, —O-aryl, —O-(alkylaryl), glycerol, glycerol monoester or diester, —NH₂, —NH(alkyl), —N(alkyl)₂, —NH(aryl), —N(aryl)₂, —NH(alkylaryl), —N(alkylaryl)₂, —SH, —S-alkyl, —S-aryl, or —S-(alkylaryl),
L² is a group selected from —(C═O)— or —(CH₂)—,
V and W are groups independently selected from methylene, alkenediyl, or substituted para-phenylenediamine,
and wherein d+e+f is from 9 to 17.
the number of methylene groups is from 8 to 16,
the number of substituted para-phenylenediamine groups is from 0 to 3, and
the number of alkenediyl groups is from 0 to 3.

Embodiment II. The compound of Embodiment I, wherein R¹ is a glycerol diester group, L¹ is —(C═O)—, the number of methylene groups is 16, the number of substituted para-phenylenediamine group is 1, the number of alkenediyl group is 0, p is 1, q is 0, and R² is a phenyl group.

Embodiment III. The compound of Embodiment I, wherein R¹ is a glycerol diester group, L¹ is —(C═O)—, the number of methylene groups is 15, the number of substituted para-phenylenediamine groups is 2, the number of alkenediyl group is 0, p is 1, q is 0, and R² is a phenyl group.

Embodiment IV. The compound of Embodiment I, wherein R¹ is a glycerol diester group, L¹ is —(C═O)—, the number of methylene groups is 14, the number of substituted para-phenylenediamine groups is 1, the number of alkenediyl group is 1, p is 1, q is 0, and R² is a phenyl group.

Embodiment V. The compound of Embodiment I, wherein R¹ is a glycerol diester group, L¹ is —(C═O)—, the number of methylene groups is 13, the number of substituted para-phenylenediamine groups is 2, the number of alkenediyl group is 1, p is 1, q is 0, and R² is a phenyl group.

Embodiment VI. The compound of Embodiment I, wherein R¹ is a —OH group, L¹ is —(C═O)—, the number of methylene groups is 16, the number of substituted para-phenylenediamine groups is 1, the number of alkenediyl group is 0, p is 1, q is 0, and R² is a methyl group.

Embodiment VII. The compound of Embodiment I, wherein R¹ and R³ are —OCH₃ groups, L¹ and L² are —(C═O)— groups, the number of methylene groups is 33, the number of substituted para-phenylenediamine group is 1, the number of alkenediyl group is 0, p is 1, and q is 1.

Embodiment VIII. An antidegradant product made by a process comprising:
hydroformylating one or more of:
a fatty acid or fatty acid derivative comprising one or more of a straight chain fatty acid having one or more unsaturations and from 10 to 20 carbon atoms, or an amide or thioester or thiocarboxylic acid thereof, or a mono-, di-, or triglyceride thereof, or an alkyl ester or an aryl ester or an alkylaryl ester thereof wherein the alkyl or aryl or alkylaryl group has from one to twelve carbon atoms, or
a fatty alcohol comprising one or more of a straight chain fatty alcohol having one or more unsaturations and from 10 to 20 carbon atoms,
by reaction with carbon monoxide and hydrogen gases to obtain a hydroformylated intermediate; and
functionalizing the hydroformylated intermediate by reaction with a substituted or unsubstituted paraphenylene diamine to obtain the antidegradant product.

Embodiment IX. The antidegradant product of Embodiment VIII, wherein the fatty acid or fatty acid derivative comprises soybean oil.

Embodiment X. The antidegradant product of Embodiment VIII, wherein the fatty acid or fatty acid derivative comprises one or more of soybean oil, olive oil, canola oil, corn oil, cottonseed oil, grapeseed oil, flax oil, hempseed oil, peanut oil, or sunflower oil.

Embodiment XI. The antidegradant product of Embodiment VIII, wherein the fatty acid or fatty acid derivative comprises a mono-, di-, or triglyceride of a residue of one or more of palmitic acid, stearic acid, oleic acid, linoleic acid, or linolenic acid.

Embodiment XII. The antidegradant product of Embodiment VIII, wherein the fatty alcohol comprises one or more of palmitic alcohol, stearic alcohol, oleic alcohol, linoleic alcohol, or linolenic alcohol.

Embodiment XIII. An antidegradant product represented by the following formula:

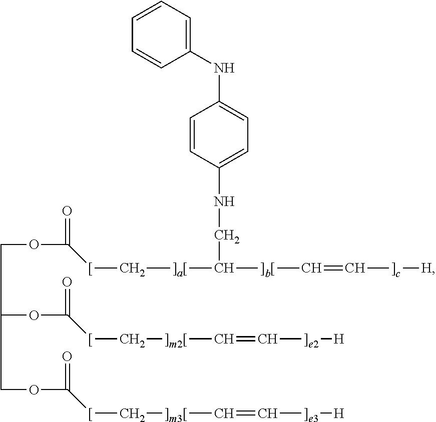

wherein m2 is from 11 to 17, e2 is from 0 to 3, m3 is from 11 to 17, e3 is from 0 to 3, and the groups —CH₂— and —CH═CH— can be in any order, and a+b+c is from 9 to 17.

Embodiment XIV. An antidegradant product represented by the following formula:

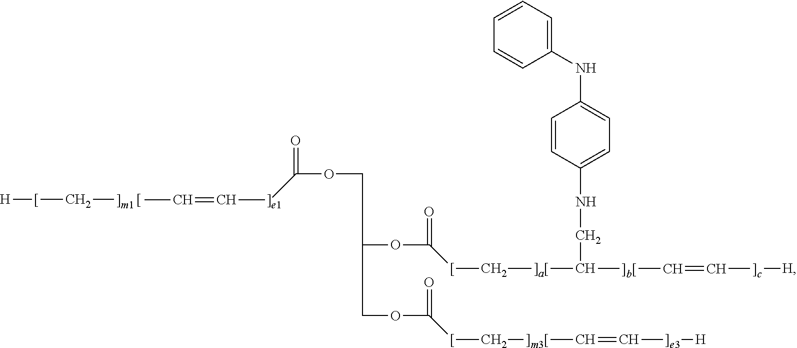

wherein m1 is from 11 to 17, e1 is from 0 to 3, m3 is from 11 to 17, e3 is from 0 to 3, and the groups —CH₂— and —CH═CH— can be in any order, and a+b+c is from 9 to 17.

Embodiment XV. An antidegradant product represented by the following formula:

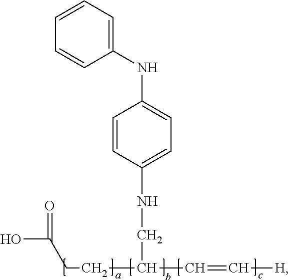

wherein a+b+c is from 9 to 17.

Embodiment XVI. An antidegradant product represented by the following formula:

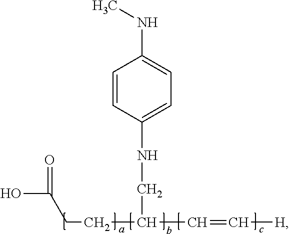

wherein a+b+c is from 9 to 17.

Embodiment XVII. An antidegradant product represented by the following formula:

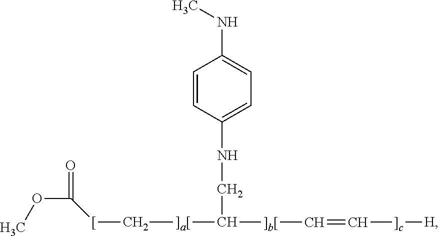

wherein a+b+c is from 9 to 17.

Embodiment XVII. An antidegradant product represented by the following formula:

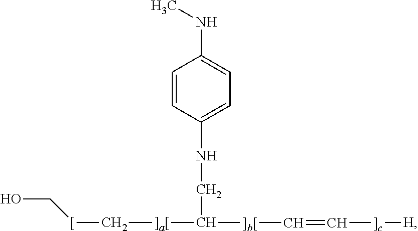

wherein a+b+c is from 9 to 17.

Embodiment XIX. An antidegradant product represented by the following formula:

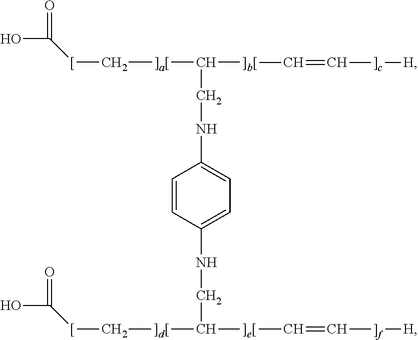

wherein a+b+c is from 9 to 17 and d+e+f is from 9 to 17.

Embodiment XX. An antidegradant product represented by the following formula:

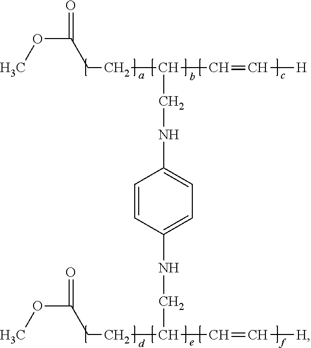

wherein a+b+c is from 9 to 17 and d+e+f is from 9 to 17.

Embodiment XXI. An antidegradant product represented by the following formula:

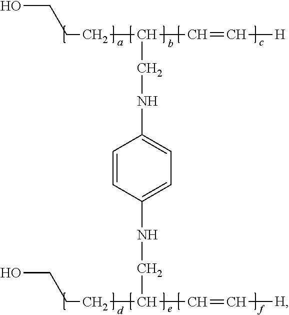

wherein a+b+c is from 9 to 17 and d+e+f is from 9 to 17.

### Example 1-1: Synthesis of Hydroformylated Soybean Oil with Manual Syngas Addition

Commercially available soybean oil (100 grams, Sigma Aldrich lot #MKCK 7562), 0.7 g (2.7 mmol) triphenylphosphine ligand, 50 mL anhydrous toluene, and 7.4 g of 7000 ppm Rh(CO)₂(acac) solution (0.5 mmol Rh) in toluene were combined in a 300 mL Hastelloy C autoclave. After proper closure and installation in the autoclave stand inside of a fume hood, the autoclave was pressure purged three times with nitrogen to remove any oxygen from the head space and feed lines and then charged to 200 psig with 1:1 H2:CO gas. A target stir rate of 750 rpm was set, and the autoclave was heated to a target temperature of 100° C. Upon reaching the desired targets, pressure was maintained in the range of 280-320 psig by manual addition of 1:1 H2:CO gas as needed. After 10 hours, the autoclave was cooled to ambient temperature and vented to remove excess pressure. The full autoclave contents were transferred to a round bottom flask and concentrated via rotary evaporation to remove most of the toluene to give hydroformylated soybean oil batch #1449-25, which was then characterized via 1H NMR spectroscopy. For this batch, the determination of aldehyde content was 2.43 moles aldehyde per mole triglyceride, which equates to 53% conversion of the available olefin groups. The common hydrogenation side reaction (i.e., H2 addition to an olefin) is also evaluated via NMR but was found to be negligible for this batch (0.01 moles hydrogenated olefin per mole triglyceride). Hydroformylated soybean oil batch #1449-25 was carried forward to synthesis presented in example 2-1 below without further modification.

### Example 1-2: Synthesis of Hydroformylated Soybean Oil Using Automatic Pressure Control

Commercially available soybean oil (100 grams, Sigma Aldrich lot #MKCK 7562), 0.7 g (2.7 mmol) triphenylphosphine ligand, 50 mL anhydrous toluene, and 6.5 g of 7850 ppm rhodium bis-2-ethylhexanoate solution in Texanol™ (0.5 mmol Rh) were combined in a 300 mL stainless steel autoclave. After proper closure and installation in the autoclave stand, the autoclave was pressure purged three times with nitrogen to remove any oxygen from the head space and feed lines and then charged to 600 psig with 1:1 H2:CO gas. A target stir rate of 750 rpm was set, and the autoclave was heated to a target temperature of 80° C. Upon reaching the desired targets, pressure was maintained in the range of 580-620 psig by addition of 1:1 H2:CO gas automatically via pressure control loop. After 5 hours, the autoclave was cooled to ambient temperature and vented to remove excess pressure. The full autoclave contents were transferred to a round bottom flask and concentrated via rotary evaporation to remove most of the toluene to give hydroformylated soybean oil batch #1449-28, which was then characterized via 1H NMR spectroscopy. For this batch, the determination of aldehyde content was 0.97 moles aldehyde per mole triglyceride, which equates to 21% conversion of the available olefin groups. The common hydrogenation side reaction (i.e., H2 addition to an olefin) is also evaluated via NMR and found to be 0.26 moles hydrogenated olefin per mole triglyceride or 10% conversion of the available olefin groups.

### Example 2-1: Synthesis of the Imine-Containing Intermediate Via Condensation of Hydroformylated Soybean Oil with 4-ADPA in Toluene

Hydroformylated soybean oil batch #1449-25 (1.57 g; about 4.0 mmol aldehyde) was treated with 4-ADPA (0.83 g; 4.5 mmol) in toluene (6 mL) at 70 deg C. under vacuum distillation conditions (volatiles were removed in less than 15 minutes). The resulting dark oily liquid was dissolved in deuterated chloroform, and the obtained solution was characterized by proton and carbon 13 NMR spectroscopy. The obtained proton and carbon 13 spectra both indicated complete disappearance of the aldehyde peaks and presence of the desired imine peaks.

### Example 2-2: Synthesis of the Imine-Containing Intermediate Via Condensation of Hydroformylated Soybean Oil with 4-ADPA in Isopropanol

Hydroformylated soybean oil batch #1449-48 (1.039 g; 2.348 mmol aldehyde) was treated with 4-ADPA (0.4455 g; 2.418 mmol) in isopropanol (3 mL) at room temperature for 6 minutes. An aliquot of the dark solution was taken out and characterized directly by proton and carbon 13 NMR spectroscopy. The obtained proton and carbon 13 spectra both indicated complete disappearance of the aldehyde peaks and presence of the desired imine peaks.

### Example 3-1: Synthesis of 4-ADPA Functionalized with Soybean Oil Via Direct Reductive Alkylation of Hydroformylated Soybean Oil and 4-ADPA with Sodium Triacetoxyborohydride in Isopropanol

To a 500-mL round bottom flask fitted with a stir-bar were charged hydroformylated soybean oil batch #1449-27 (38.8 g; 45.0 mmol aldehyde), 4-ADPA (8.22 g; 44.6 mmol), and isopropanol (230 mL). The mixture was protected with N₂ blanket and stirred at room temperature. About 24 minutes later, sodium triacetoxyborohydride (10.3 g; 48.6 mmol) was loaded in one portion. The reaction mixture was stirred at room temperature for 4 hours. DI water (60 mL) was added portion-wise (no exothermic event was observed). Saturated aqueous NaCl (100 mL) was added. Product was extracted with AcOEt (190 mL). The aqueous phase was discarded. The organic phase was washed with aqueous sodium hydroxide (9.5 g NaOH dissolved in 370 mL DI water) and saturated aqueous NaCl (150 mL), then stripped of volatiles under reduced pressure (rotary evaporator; water bath=55 deg C.). The resulting dark oil was dried on the rotary evaporator at 55 deg C. until constant weight. Yield=42.7 g (92% of the theoretical) as a dark oily liquid. An aliquot of the product was dissolved in deuterated chloroform, and the obtained solution was characterized by proton and carbon 13 NMR spectroscopy. The obtained proton and carbon 13 spectra both indicated complete disappearance of the aldehyde and imine peaks, as well as presence of the desired secondary amine peaks.

### Example 3-2: Synthesis of 4-ADPA Functionalized with Soybean Oil Via Direct Reductive Alkylation of Hydroformylated Soybean Oil and 4-ADPA Under Hydrogenation Conditions in Toluene

A 300 mL Paar autoclave was charged with 4-ADPA (7.8 g), hydroformylated soybean oil batch #1449-46 (21.0 g), toluene (153 mL), and 3% platinum on carbon 62% water (9.0 g). The autoclave was sealed, then purged with 20 PSI nitrogen gas. The vessel was charged with 100 PSI H2, stirred for 15 minutes at RT, then vented. The content of the autoclave was heated to 150° C. When the target temperature was reached, 500 PSI H2 was continuously fed into the vessel for 4 hours. Upon cooling to room temperature, the reaction contents were filtered through a 0.5 uM frit to separate catalyst residues from the product. The filtrate was stripped of volatiles under reduced pressure (rotary evaporator; water bath=65° C.). An aliquot of the product was dissolved in deuterated chloroform, and the obtained solution was characterized by proton and carbon 13 NMR spectroscopy. The obtained proton and carbon 13 spectra both indicated complete disappearance of the aldehyde and imine peaks, as well as presence of the desired amine peaks.

### Example 4-1: Synthesis of 4-ADPA Functionalized with Fatty Acids Via Saponification of 4-ADPA Functionalized with Soybean Oil in Hot Isopropanol Followed by Acidic Workup (Small Scale)

To a 50-mL round bottom flask fitted with a stir-bar and a reflux condenser were loaded soybean oil functionalized with 4-ADPA batch #5048-10 (about 2.5 g), isopropanol (15 mL), and sodium hydroxide (0.4565; 11.4 mmol). The reaction mixture was stirred under N₂ blanket and refluxed (oil bath temperature=95 deg C.) for 8 hours, then allowed to cool to room temperature overnight. The resulting mass was triturated with isopropanol (30 mL) to loosen up the solid. The latter was collected by vacuum filtration (Buchner—filter paper), then quickly rinsed with isopropanol (2×10 mL). The solid was dissolved in DI water (50 mL). The obtained dark solution was acidified by slow addition of aqueous 37% HCl until pH=2.5-3.0 (pH paper). The formed sticky material (product) was extracted with ethyl acetate (50 mL). The aqueous phase was discarded. The organic phase was washed three times with a mixture of DI water and saturated NaCl (20 mL+10 mL respectively), then stripped of volatiles under reduced pressure (rotary evaporator; water bath=55 deg C.). The resulting sludge was dried on the rotary evaporator (water bath=55 deg C.) until constant weight. Yield=1.4 g as a waxy dark solid. Proton NMR spectroscopy showed that the level of 4-ADPA functionalization in the product was lower than prior saponification. Some of the 4-ADPA moieties were hydrolyzed then lost during purification. This undesirable side-reaction is the reason why the isolated yield is low.

### Example 4-2: Synthesis of 4-ADPA Functionalized with Fatty Acids Via Saponification of 4-ADPA Functionalized with Soybean Oil in Hot Isopropanol Followed by Acidic Workup (Scale-Up to 1-L Reactor Size)

To a 1-L reactor equipped with overhead stirred, temperature probe and reflux condenser were loaded 4-ADPA functionalized with soybean oil (about 1 mol 4-ADPA/mol triglyceride) in solution in isopropanol (196 g of solution containing 33.7 g of 4-ADPA functionalized with soybean oil; about 31.1 mmol triglyceride). Sodium hydroxide (8.0 g; 200 mmol) was added in one portion. The reaction mixture was stirred under N2 blanket and refluxed for 8 hours, then allowed to cool to room temperature overnight. Additional isopropanol (420 mL) was added and stirring was applied to loosen up the solid. The solid was collected by vacuum filtration (Buchner—filter paper), and quickly rinsed with IPA (2×140 mL). The solid was collected and dissolved in DI water (700 mL) in a large beaker under magnetic stirring. The obtained dark solution was acidified by slow addition of aqueous 37% HCl until pH=2.5-3.5 (pH paper). The formed sticky material (product) was extracted with ethyl acetate (2×350 mL). The aqueous phase was discarded. The combined organic phases were washed with aqueous 240 g/L sodium chloride (3×420 mL), then stripped of volatiles under reduced pressure (rotary evaporator; water bath=55 deg C.). The resulting sludge was dried on the rotary evaporator (water bath=55 deg C.) for 3-4 hours. Yield=11.65 grams as a waxy dark solid. An aliquot of product was dissolved in CDCl3 and the obtained solution was characterized by proton and carbon 13 NMR spectroscopy. The obtained proton and carbon 13 spectra both indicated absence of glycerol peaks, complete disappearance of the ester peaks, and presence of the desired carboxylic acid and secondary amine peaks. Proton NMR spectroscopy showed that the level of 4-ADPA functionalization in the product was lower than prior saponification. Some of the 4-ADPA moieties were hydrolyzed then lost during purification. This undesirable side-reaction is the reason why the isolated yield is low.

### Example 5: Synthesis of Fatty Acids Via Saponification of Soybean Oil

To a round bottom flask fitted with stir-bar and reflux condenser were charged soybean oil from Sigma part #S7381 (90.7 g; MW=about 880 g/mol; about 103 mmol triglyceride), sodium hydroxide (14.78 g; 369.5 mmol), and isopropanol (570 mL). The reaction mixture was stirred under N2 protection and refluxed (oil bath temperature=98 deg C.; careful: a brief exothermic event occurred during the temperature ramp) for 5 hours (a soft solid separated). Upon cooling to room temperature (overnight), the soft solid was collected by vacuum filtration (Buchner—filter paper), and rinsed with isopropanol (3×50 mL). The damp soft solid (volume filtrate=450 mL; 62.5% solvent recovery) was dissolved in DI water (150 mL) under gentle magnetic stirring. The obtained light-orange solution was acidified with aqueous 37% HCl until pH<2 (a cold tap water bath was used to mitigate the exothermic neutralization reaction). The product was extracted with AcOEt (250 mL). The aqueous layer was discarded. The organic layer was washed with DI water (2×100 mL), dried over anhydrous MgSO4, filtered, then stripped of volatiles under reduced pressure (rotary evaporator; water bath=50 deg C.). The resulting orange liquid was dried on the rotary evaporator under reduced pressure (<15 mbars) for 3 hours. Yield=73.5 g (85% of the theoretical) as a light-orange liquid. Product was stored in the refrigerator, whereupon it turned into a waxy solid. An aliquot of product was dissolved in CDCl3 and the obtained solution was characterized by proton and carbon 13 NMR spectroscopy. The obtained proton and carbon 13 spectra both indicated absence of glycerol peaks, complete disappearance of the ester peaks, and presence of the desired carboxylic acid peaks.

### Example 6: Synthesis of Hydroformylated Fatty Acids Via Hydroformylation of Unsaturated Fatty Acids

The hydroformylation of soybean oil was examined under a wide variety of operating conditions and catalysts at the Longview, TX site. For illustrative purposes, the preparation of hydroformylated fatty acid batch #5204-03 is described below.

In an inert atmosphere glove box, 40.01 grams of soybean fatty acid (batch 5048-18 prepared at the Akron, OH site), 0.7 g (2.7 mmol) triphenylphosphine ligand, 50 mL anhydrous n-hexane, and 6.5 g of 7850 ppm rhodium bis(2-ethylhexanoate) solution (0.5 mmol Rh) in Texanol™ were combined in a 300 mL stainless steel autoclave. After proper closure and installation in the autoclave stand, the autoclave was pressure purged three times with nitrogen to remove any oxygen from the head space and feed lines and then charged to 200 psig with 1:1 H2:CO gas. A stir rate of 750 rpm was set, and the autoclave was heated to a target temperature of 70° C. Upon reaching the desired targets, pressure was maintained in the range of 280-320 psig by automatic addition of 1:1 H2:CO gas as needed. After 24 hours, the autoclave was cooled to ambient temperature and vented to remove excess pressure. The full autoclave contents were transferred to a round bottom flask and concentrated via rotary evaporation to remove most of the n-hexane to give hydroformylated fatty acid batch #5204-03, which was then shipped to the Kingsport, TN site for characterization via 1H NMR spectroscopy. For this batch, the determination of aldehyde content was 0.92 moles aldehyde per mole fatty acid.

### Example 7: Synthesis of 4-MAPA Functionalized with Fatty Acids Via Direct Reductive Alkylation of Hydroformylated Fatty Acids and 4-MAPA with Sodium Triacetoxyborohydride in Isopropanol

To a 500-mL round bottom flask fitted with a stir-bar were loaded hydroformylated fatty acids batch 5204-03 (15.4 g; about 49 mmol aldehyde), and isopropanol (90 mL). The mixture was stirred under N2 protection. A solution of N-methyl-4-aminoaniline batch 4795-80 (6.3 g; 51.5 mmol) in isopropanol (20 mL) was added and the reaction mixture was stirred at room temperature for about 10 minutes to form the imine intermediate. Sodium triacetoxyborohydride “STAB” (12.5 g; 59 mmol) was loaded in one portion. The reaction mixture was stirred at room temperature for 4 hours. Saturated aqueous NaCl (200 mL) was slowly added to the flask and the mixture was stirred for a few minutes. The mixture was transferred to a 1-L separatory funnel containing ethyl acetate (250 mL). The separatory funnel was shaken. After decantation, the aqueous layer (bottom) was discarded. The organic layer (top) was washed with saturated aqueous NaHCO₃ (2×100 mL) and saturated NaCl (100 mL), then stripped of volatiles under reduced pressure (rotary evaporator; water bath=50 deg C.). The resulting material was dried under vacuum at 50 deg C. overnight. Yield=15.5 g as a waxy dark solid. An aliquot of product was dissolved in CDCl3 and the obtained solution was characterized by proton and carbon 13 NMR spectroscopy. The obtained proton and carbon 13 spectra both indicated complete disappearance of the aldehyde peaks, no imine peaks, and presence of the desired secondary amine peaks.

In order to demonstrate the antioxidant efficacy of Compounds of the Disclosure, the oxidative induction time (OIT) of selected examples were evaluated. OIT is measured according to a procedure carried out in a differential scanning calorimeter (DSC) and is used by those of ordinary skill in the art to predict thermo-oxidative performance of a material. In this procedure, each sample to analyze is held in a sample cell and heated under a nitrogen atmosphere to a preselected temperature. Oxygen is then introduced to the sample cell and the length of time before the onset of degradation, as seen by the initiation of increase in heat flow, is measured. N-(1,3-dimethylbutyl)-N′-phenyl-para-phenylenediamine (6PPD), a known antidegradant additive for rubber that is commercially available from Eastman Chemical Company under the trademark Santoflex, and (unmodified) soybean oil purchased from Sigma-Aldrich were also tested as controls for OIT. The OIT results are presented in the following tables:

TABLE 1
Oxidative induction time (OIT) measured
at 150 deg C. in polyisoprene matrix

| | | Level of 4- | |
|---|---|---|---|
| | | ADPA contained | |
| | Additive | in additive | OIT |
| | loading | (mol 4-ADPA/ | results |
| Example | (phr) | mol additive) | (min) |
| Blank control (no additive) | 0.0 | 0.0 | 2.2 |
| Control # 1 (soybean oil) | 2.0 | 0.0 | 1.8 |
| Control # 2 (6PPD) | 0.5 | 1.0 | 48.7 |
| 3-1 (4-ADPA-triglyceride form) | 0.5 | 1.0 | 223.3 |
| 3-1 (4-ADPA-triglyceride form) | 1.3 | 1.0 | 264.3 |
| 3-2 (4-ADPA-triglyceride form) | 0.5 | 1.9 | 106.3 |
| 4-1 (4-ADPA-fatty acid form) | 0.5 | <2.14 | 161.9 |

TABLE 2
Oxidative induction time (OIT) measured
at 160 deg C. in polyisoprene matrix

| | | Level of 4- | |
|---|---|---|---|
| | | ADPA contained | |
| | Additive | in additive | OIT |
| | loading | (mol 4-ADPA/ | results |
| Example | (phr) | mol additive) | (min) |
| Blank control (no additive) | 0.0 | 0.0 | 1.14 |
| Control # 1 (soybean oil) | 2.0 | 0.0 | 1.3 |
| Control # 2 (6PPD) | 0.5 | 1.0 | 22.7 |
| 3-1 (4-ADPA-triglyceride form) | 0.5 | 1.0 | 129.9 |
| 3-1 (4-ADPA-triglyceride form) | 1.3 | 1.0 | 170.8 |
| 3-2 (4-ADPA-triglyceride form) | 0.5 | 1.9 | 197.9 |
| 4-1 (4-ADPA-fatty acid form) | 0.5 | <2.14 | 57.0 |

TABLE 3
Oxidative induction time (OIT) measured
at 150 deg C. in LSBR matrix

| | | Level of 4- | |
|---|---|---|---|
| | | ADPA contained | |
| | Additive | in additive | OIT |
| | loading | (mol 4-ADPA/ | results |
| Example | (phr) | mol additive) | (min) |
| Blank control (no additive) | 0.0 | 0.0 | 8.1 |
| Control # 2 (6PPD) | 0.5 | 1.0 | 113.0 |
| 3-1 (4-ADPA-triglyceride form) | 0.5 | 1.0 | 472 |
| 3-2 (4-ADPA-triglyceride form) | 0.5 | 1.9 | 587 |
| 4-1 (4-ADPA-fatty acid form) | 0.5 | <2.14 | 395.8 |

TABLE 4
Oxidative induction time (OIT) measured
at 160 deg C. in LSBR matrix

| | | Level of 4- | |
|---|---|---|---|
| | | ADPA contained | |
| | Additive | in additive | OIT |
| | loading | (mol 4-ADPA/ | results |
| Example | (phr) | mol additive) | (min) |
| Blank control (no additive) | 0.0 | 0.0 | 6.0 |
| Control # 2 (6PPD) | 0.5 | 1.0 | 54.0 |
| 3-1 (4-ADPA-triglyceride form) | 0.5 | 1.0 | 253.0 |
| 4-1 (4-ADPA-fatty acid form) | 0.5 | <2.14 | 154.3 |

TABLE 5
Oxidative induction time (OIT) measured
at 150 deg C. in polybutadiene matrix

| | | Level of 4- | |
|---|---|---|---|
| | | ADPA contained | |
| | Additive | in additive | OIT |
| | loading | (mol 4-ADPA/ | results |
| Example | (phr) | mol additive) | (min) |
| Blank control (no additive) | 0.0 | 0.0 | 3.5 |
| Control # 2 (6PPD) | 0.5 | 1.0 | 42.4 |
| 3-1 (4-ADPA-triglyceride form) | 0.5 | 1.0 | 124.0 |
| 3-2 (4-ADPA-triglyceride form) | 0.5 | 1.9 | 144.0 |
| 4-1 (4-ADPA-fatty acid form) | 0.5 | <2.14 | 154.8 |
| 4-1 (4-ADPA-fatty acid form) | 1.3 | <2.14 | 260.6 |

TABLE 6
Oxidative induction time (OIT) measured
at 160 deg C. in polybutadiene matrix

| | | Level of 4- | |
|---|---|---|---|
| | | ADPA contained | |
| | Additive | in additive | OIT |
| | loading | (mol 4-ADPA/ | results |
| Example | (phr) | mol additive) | (min) |
| Blank control (no additive) | 0.0 | 0.0 | 2.0 |
| Control # 1 (soybean oil) | 0.5 | 0 | 2.6 |
| Control # 2 (6PPD) | 0.5 | 1.0 | 31.0 |
| 3-1 (4-ADPA-triglyceride form) | 0.5 | 1.0 | 70.1 |
| 3-2 (4-ADPA-triglyceride form) | 0.5 | 1.9 | 66.2 |
| 4-1 (4-ADPA-fatty acid form) | 0.5 | <2.14 | 44.4 |
| 4-1 (4-ADPA-fatty acid form) | 1.3 | <2.14 | 113.2 |

As indicated by the above data, Compounds of the Disclosure demonstrate excellent antioxidant performance that compares well to 6PPD and indicates utility in rubber and other applications that can benefit from a highly active antioxidant compound.

1. A compound represented by formula I: 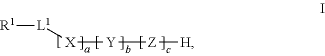
wherein
R¹ is a group selected from —OH, —O-alkyl, —O-aryl, —O-(alkylaryl), 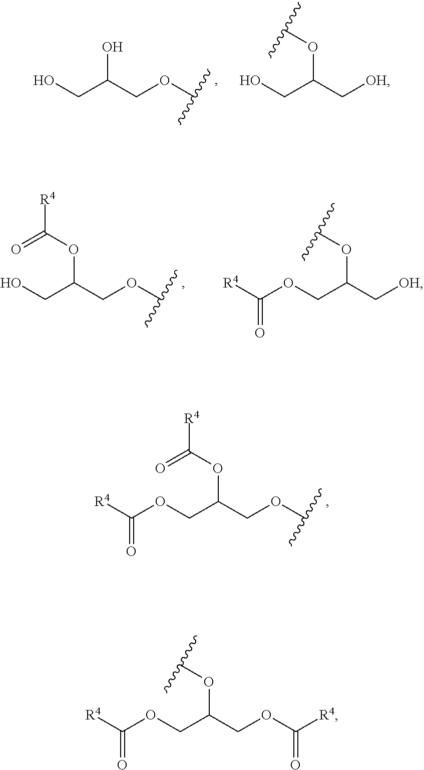
—NH₂, —NH(alkyl), —N(alkyl)₂, —NH(aryl), —N(aryl)₂, —NH(alkylaryl), —N(alkylaryl)₂, —SH, —S-alkyl, —S-aryl, and —S-(alkylaryl),
each R⁴ is independently selected from the group consisting of C₅₋₂₆ alkyl and C₅₋₂₆ alkenyl,
L¹ is a group selected from —(C═O)— and —(CH₂)—,
a+b+c is an integer from 9 to 17,
each X, each Y, and each Z are groups independently selected from methylene, alkenediyl, and a substituted para-phenylenediamine having formula II: 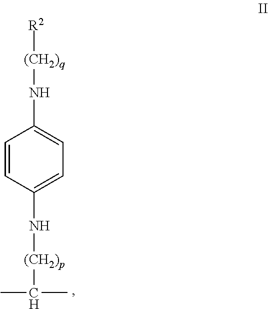
wherein
each p is independently 0 or 1,
each q is independently 0 or 1,
each R² is a group independently selected from —H, -alkyl, -aryl, -alkylaryl, -arylalkyl, and a group having formula III: 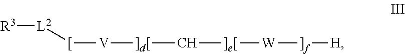
wherein
each R³ is a group independently selected from —OH, —O-alkyl, —O-aryl, —O-(alkylaryl), 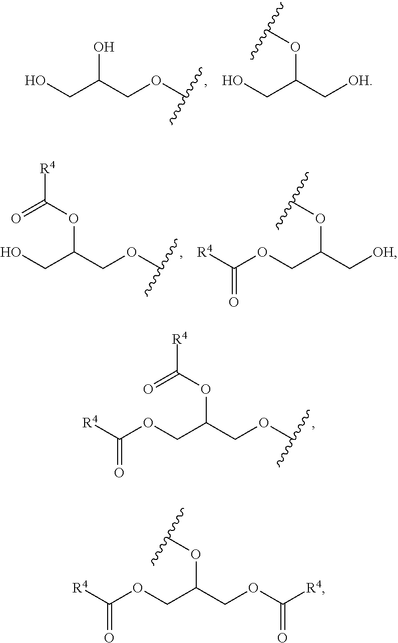
-NH₂, —NH(alkyl), —N(alkyl)₂, —NH(aryl), —N(aryl)₂, —NH(alkylaryl), —N(alkylaryl)₂, —SH, —S-alkyl, —S-aryl, and —S-(alkylaryl),
each L² is a group independently selected from —(C═O)— and —(CH₂)—,
each V and each W are groups independently selected from methylene, alkenediyl, and a substituted para-phenylenediamine having formula II-A: 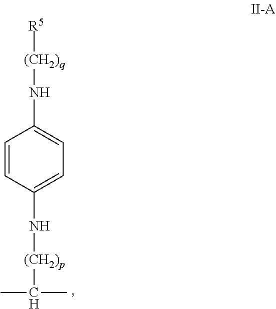
wherein each R⁵ is a group independently selected from —H, -alkyl, -aryl, -alkylaryl, and -arylalkyl,
and wherein d+e+f is an integer from 9 to 17.

2. The compound of claim 1, wherein R¹ is 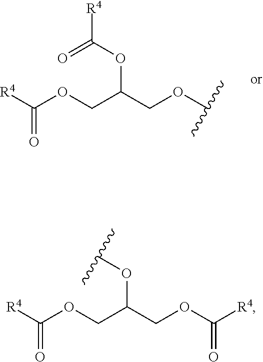
L¹ is —(C═O)—, the total number of substituted para-phenylenediamine groups having formula II and/or formula IIa in the compound is 1, p is 1, q is 0, and R² is a phenyl group.

3. The compound of claim 1, wherein R¹ is 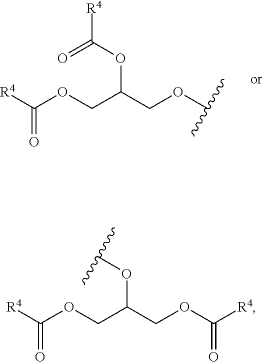
L¹ is —(C═O)—, the total number of substituted para-phenylenediamine groups having formula II and/or formula IIa in the compound is 2, p is 1, q is 0, and R² is a phenyl group.

4. The compound of claim 1, wherein R¹ is 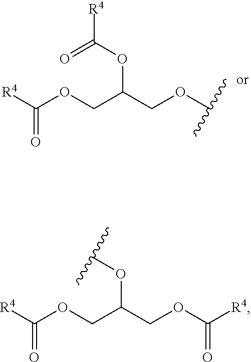
L¹ is —(C═O)—, the total number of substituted para-phenylenediamine groups having formula II and/or formula IIa in the compound is 1, p is 1, q is 0, and R² is a phenyl group.

5. The compound of claim 1, wherein R¹ is 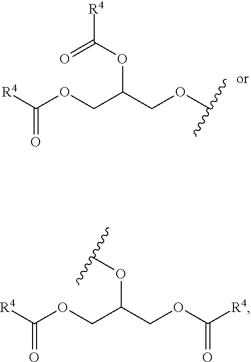
L¹ is —(C═O)—, the total number of substituted para-phenylenediamine groups having formula II and/or formula IIa in the compound is 2, p is 1, q is 0, and R² is a phenyl group.

6. The compound of claim 1, wherein R¹ is a —OH group, L¹ is —(C═O)—, the total number of substituted para-phenylenediamine groups having formula II and/or formula IIa in the compound is 1, p is 1, q is 0, and R² is a methyl group.

7. The compound of claim 1, wherein R¹ and R³ are —OCH₃ groups, L¹ and L² are —(C═O)— groups, the total number of substituted para-phenylenediamine groups having formula II and/or formula IIa in the compound is 1, p is 1, and q is 1.

8. An antidegradant product made by a process comprising:
i. hydroformylating one or more of:
1. a fatty acid or fatty acid derivative comprising one or more of a straight chain fatty acid having one or more carbon to carbon double bonds and from 10 to 20 carbon atoms, or an amide or thioester or thiocarboxylic acid thereof, or a mono-, di-, or triglyceride thereof, or an alkyl ester or an aryl ester or an alkylaryl ester thereof wherein the alkyl or aryl or alkylaryl group has from one to twelve carbon atoms, or
2. a fatty alcohol comprising one or more of a straight chain fatty alcohol having one or more carbon-to-carbon double bonds and from 10 to 20 carbon atoms,
by reaction with carbon monoxide and hydrogen gases to obtain a hydroformylated intermediate; and
ii. functionalizing the hydroformylated intermediate by reaction with a substituted or unsubstituted para-phenylene diamine having formula VII: 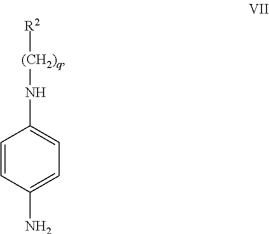
wherein q is 0 or 1,
each R² is a group independently selected from —H, -alkyl, -aryl, -alkylaryl, -arylalkyl, and a group having formula III: 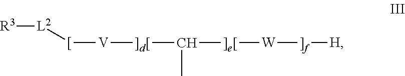
wherein each R³ is a group independently selected from —OH, —O-alkyl, —O-aryl, —O-(alkylaryl), 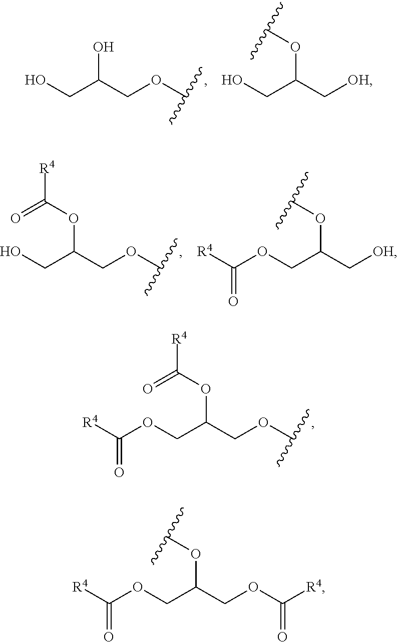
-NH₂, —NH(alkyl), —N(alkyl)₂, —NH(aryl), —N(aryl)₂, —NH(alkylaryl), —N(alkylaryl)₂, —SH, —S-alkyl, —S-aryl, and —S-(alkylaryl),
each L² is a group independently selected from —(C═O)— and —(CH₂)—, and
each R⁴ is independently selected from C₅₋₂₆ alkenyl,
to obtain the antidegradant product.

9. The antidegradant product of claim 8, wherein the fatty acid or fatty acid derivative is derived from soybean oil.

10. The antidegradant product of claim 8, wherein the fatty acid or fatty acid derivative is derived from one or more of soybean oil, olive oil, canola oil, corn oil, cottonseed oil, grapeseed oil, flax oil, hempseed oil, peanut oil, or sunflower oil.

11. The antidegradant product of claim 8, wherein the fatty acid or fatty acid derivative comprises a mono-, di-, or triglyceride of a residue of one or more of palmitic acid, stearic acid, oleic acid, linoleic acid, or linolenic acid.

12. The antidegradant product of claim 8, wherein the fatty alcohol comprises one or more of palmitic alcohol, stearic alcohol, oleic alcohol, linoleic alcohol, or linolenic alcohol.

13. The compound of claim 1 having formula X: 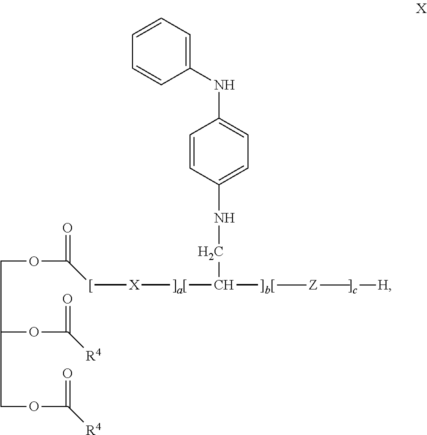
wherein each X and each Z are groups independently selected from methylene and alkenediyl and b is 1.

14. The compound of claim 1 having formula XI: 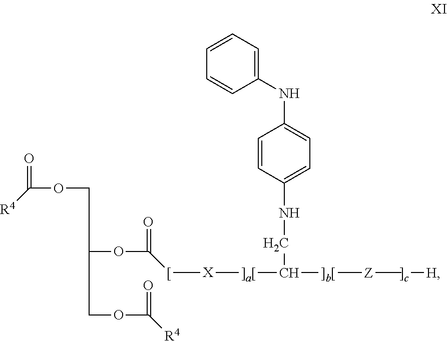
wherein each X and each Z are groups independently selected from methylene and alkenediyl and b is 1.

15. The compound of claim 1 having formula XII: 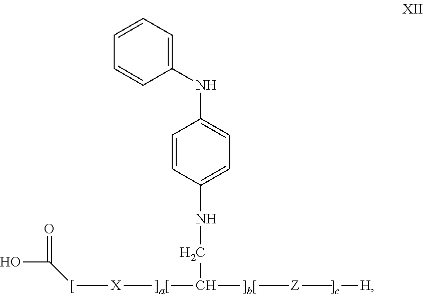
wherein each X and each Z are groups independently selected from methylene and alkenediyl and b is 1.

16. The compound of claim 1 having formula XIII: 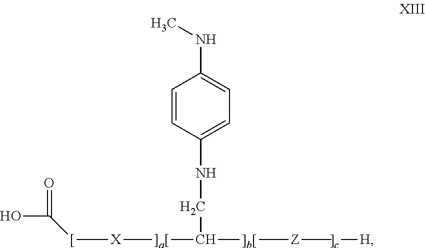
wherein each X and each Z are groups independently selected from methylene and alkenediyl and b is 1.

17. The compound of claim 1 having formula XIV: 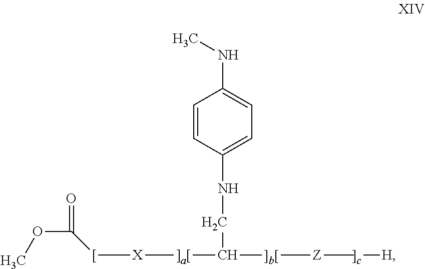
wherein each X and each Z are groups independently selected from methylene and alkenediyl and b is 1.

18. The compound of claim 1 having formula XV: 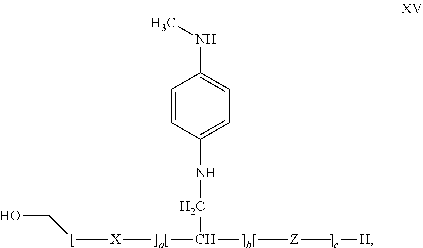
wherein each X and each Z are groups independently selected from methylene and alkenediyl and b is 1.

19. The compound of claim 1 having formula XVI: 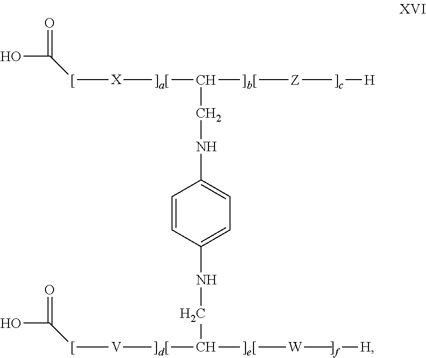
wherein each X and each Z are groups independently selected from methylene and alkenediyl and b is 1, and
wherein each V and each W are groups independently selected from methylene and alkenediyl and e is 1.

20. The compound of claim 1 having formula XVII: 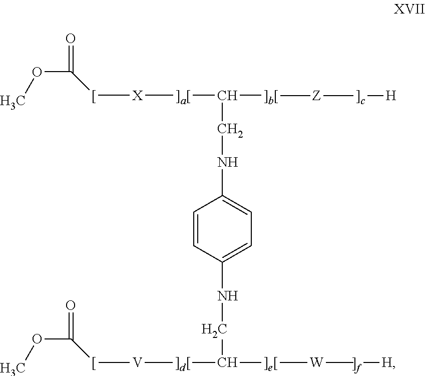
wherein each X and each Z are groups independently selected from methylene and alkenediyl and b is 1, and
wherein each V and each W are groups independently selected from methylene and alkenediyl and e is 1.

21. The compound of claim 1 having formula XVIII: 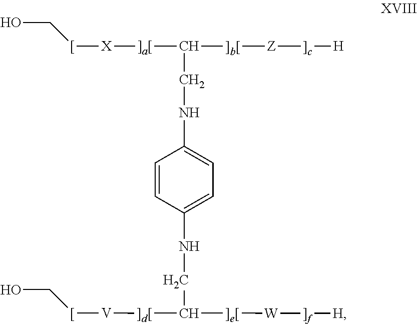
wherein each X and each Z are groups independently selected from methylene and alkenediyl and b is 1, and
wherein each V and each W are groups independently selected from methylene and alkenediyl and e is 1.

22. The compound of any one of claims 1-7 or 13-21, wherein each R⁴ is independently selected from the group consisting of:

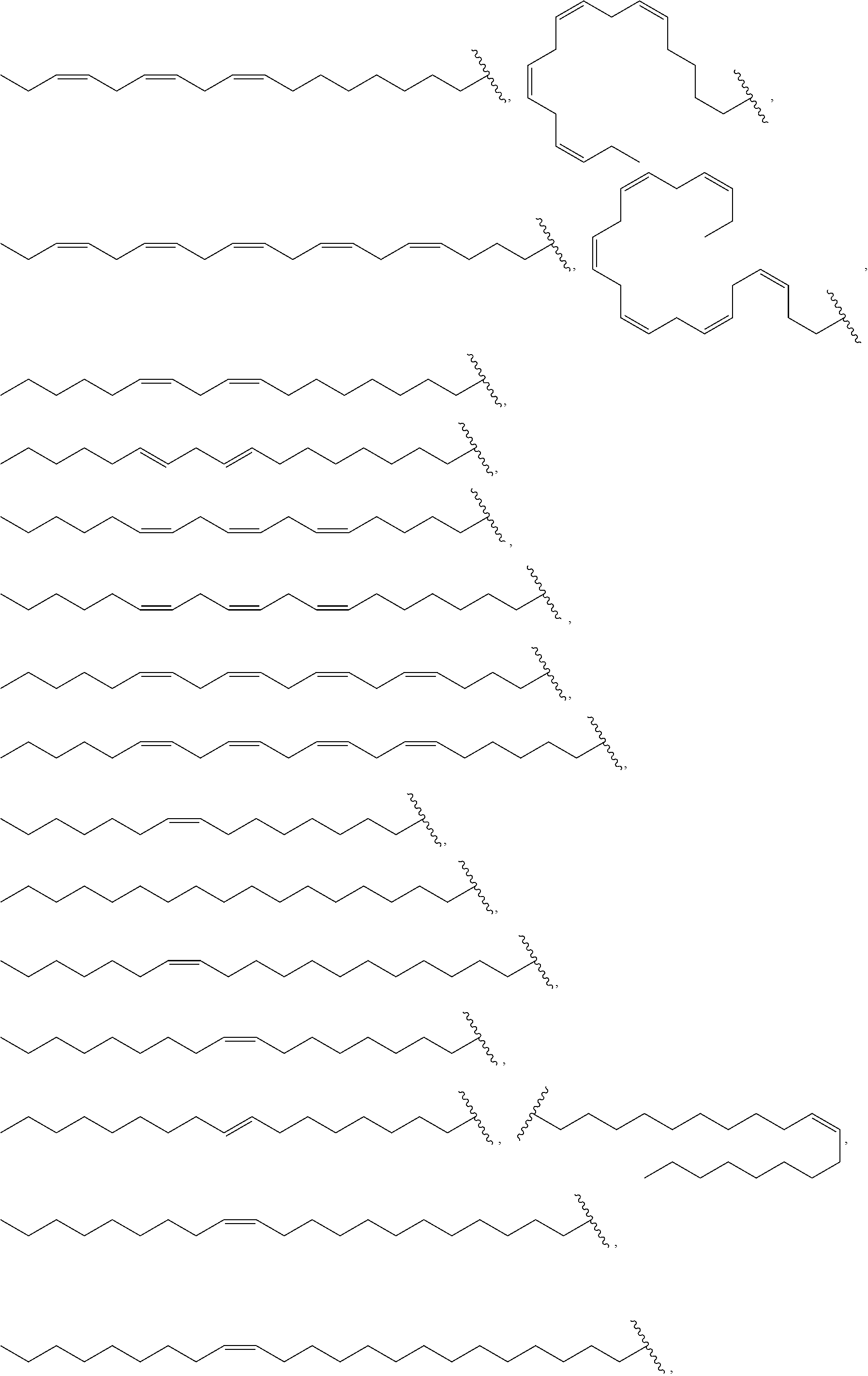

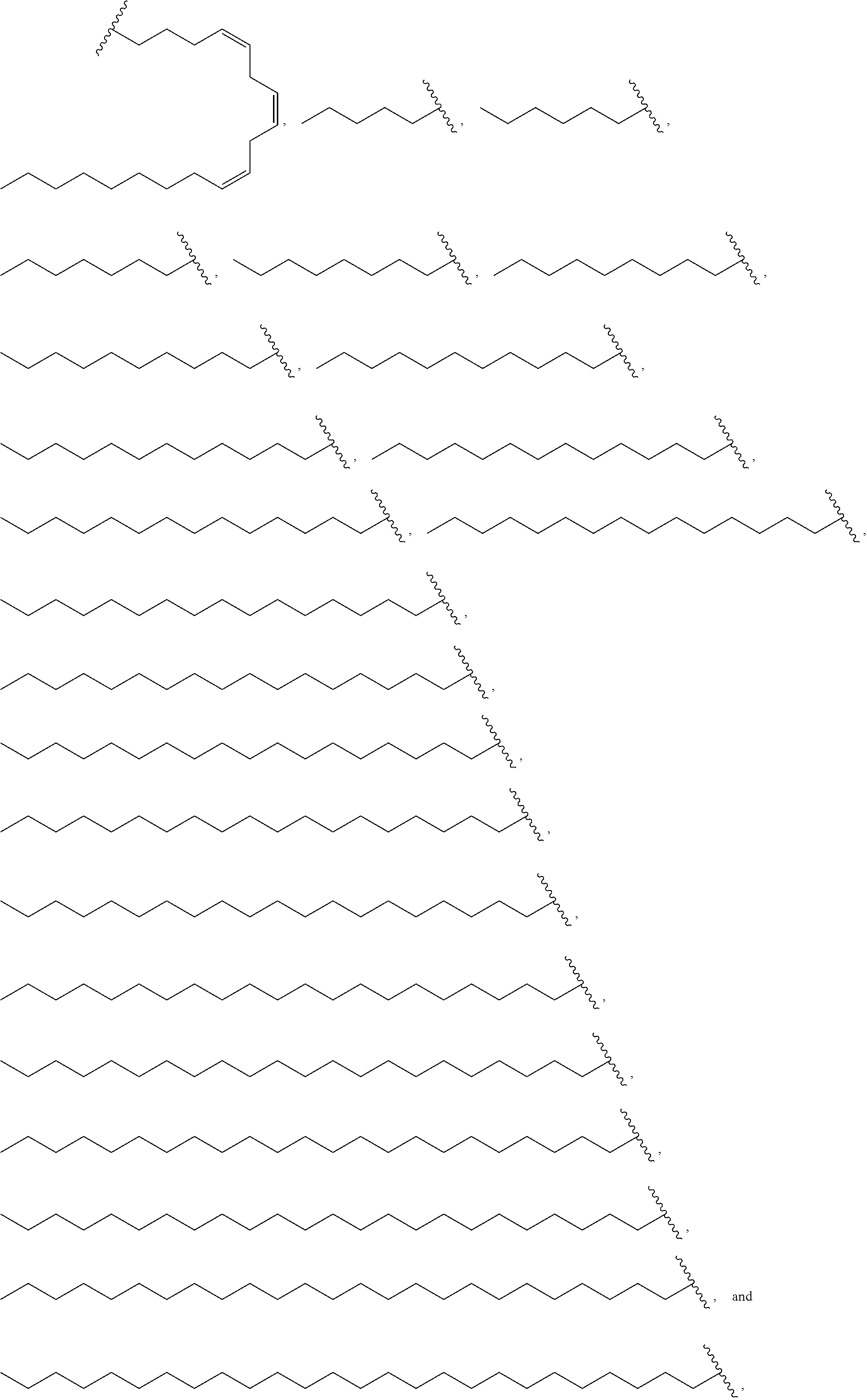

23. The compound of claim 22, wherein each R⁴ is independently selected from the group consisting of:

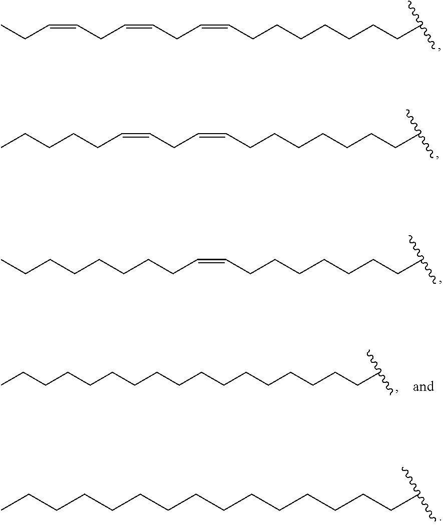 [SMILES: *CCCCCCC/C=C\C/C=C\C/C=C\CC ; *CCCCCCC/C=C\C/C=C\CCCCC ; *CCCCCCC/C=C\CCCCCCCC ; *CCCCCCCCCCCCCCCCC ; *CCCCCCCCCCCCCCC]

24. The compound of any one of claims 1-7 or 13-23, wherein a+b+c is 17.

25. The compound of any one of claims 1-7 or 13-24, wherein d+e+f is 17.
